(19) United States
(12) Patent Application Publication
(10) Pub. No.: US 2021/0398641 A1
(43) Pub. Date: Dec. 23, 2021

(54) PREDICTIVE METABOLIC INTERVENTION

(71) Applicant: UnitedHealth Group Incorporated, Minnetonka, MN (US)

(72) Inventors: Steven Catani, Athens, GA (US); Grant B. Weller, Minneapolis, MN (US); Benjamin W. Ehlert, Wayzata, MN (US); Katlyn Ann Fleming, Hopkins, MN (US)

(21) Appl. No.: 17/350,533

(22) Filed: Jun. 17, 2021

Related U.S. Application Data
(60) Provisional application No. 63/040,725, filed on Jun. 18, 2020.

(51) Int. Cl. G16H 20/30; G16H 40/67; G16H 50/20

(52) U.S. Cl. CPC G16H 20/30; G16H 50/20; G16H 40/67

(57) ABSTRACT

Various embodiments of the present invention provide methods, apparatus, systems, computing devices, computing entities, and/or the like for predictive data analysis. Certain embodiments utilize systems, methods, and computer program products that perform predictive metabolic intervention by utilizing at least one of activity recommendation machine learning models and prediction window encoding machine learning models.

### CROSS-REFERENCES TO RELATED APPLICATION(S)

The present non-provisional patent application claims priority to the U.S. Provisional Patent Application No. 63/040,725, filed on Jun. 18, 2020, which is incorporated by reference herein in its entirety.

### BACKGROUND

Various embodiments of the present invention address technical challenges related to performing metabolic intervention. Various embodiments of the present invention disclose innovative techniques for efficiently and effectively performing metabolic intervention using various predictive data analysis techniques.

### BRIEF SUMMARY

In general, embodiments of the present invention provide methods, apparatus, systems, computing devices, computing entities, and/or the like for predictive data analysis. Certain embodiments utilize systems, methods, and computer program products that perform predictive metabolic intervention by utilizing at least one of activity recommendation machine learning models and prediction window encoding machine learning models.

In accordance with one aspect, a method is provided. In some embodiments, the method comprises identifying a behavioral timeseries data object associated with a plurality of behavioral time windows; identifying a biometric timeseries data object associated with a plurality of biometric time windows; for each biometric time window, determining a desired outcome indicator based at least in part on the biometric timeseries data object; determining a plurality of activity patterns based at least in part on at least one of the behavioral timeseries data object or the biometric timeseries data object, wherein: each activity pattern is identified based at least in part on an occurrence detection time window set comprising at least one of a behavioral occurrence detection time window subset of the plurality of behavioral time windows or a biometric occurrence detection time window subset of the plurality of biometric time windows, and each activity pattern is associated with a biometric impact subset of the plurality of biometric time windows; for each activity pattern, determining an improvement likelihood measure based at least in part on each desired outcome indicator for a biometric time window that is in the biometric impact subset for the activity pattern; generating an activity recommendation machine learning model, wherein the activity recommendation machine learning model maps each activity pattern to the occurrence detection time window set for the activity pattern and the improvement likelihood measure for the activity pattern; and providing access to the activity recommendation machine learning model, wherein the activity recommendation machine learning model is configured to determine, based at least in part on an input behavioral timeseries data object and an input biometric timeseries data object, a recommended activity pattern subset of the plurality of activity patterns.

In accordance with another aspect, a computer program product is provided. The computer program product may comprise at least one computer-readable storage medium having computer-readable program code portions stored therein, the computer-readable program code portions comprising executable portions configured to identify a behavioral timeseries data object associated with a plurality of behavioral time windows; identify a biometric timeseries data object associated with a plurality of biometric time windows; for each biometric time window, determine a desired outcome indicator based at least in part on the biometric timeseries data object; determine a plurality of activity patterns based at least in part on at least one of the behavioral timeseries data object or the biometric timeseries data object, wherein: each activity pattern is identified (e.g., determined) based at least in part on an occurrence detection time window set comprising at least one of a behavioral occurrence detection time window subset of the plurality of behavioral time windows or a biometric occurrence detection time window subset of the plurality of biometric time windows, and each activity pattern is associated with a biometric impact subset of the plurality of biometric time windows; for each activity pattern, determine an improvement likelihood measure based at least in part on each desired outcome indicator for a biometric time window that is in the biometric impact subset for the activity pattern; generate an activity recommendation machine learning model, wherein the activity recommendation machine learning model maps each activity pattern to the occurrence detection time window set for the activity pattern and the improvement likelihood measure for the activity pattern; and provide access to the activity recommendation machine learning model, wherein the activity recommendation machine learning model is configured to determine, based at least in part on an input behavioral timeseries data object and an input biometric timeseries data object, a recommended activity pattern subset of the plurality of activity patterns.

In accordance with yet another aspect, an apparatus comprising at least one processor and at least one memory including computer program code is provided. In one embodiment, the at least one memory and the computer program code may be configured to, with the processor, cause the apparatus to identify a behavioral timeseries data object associated with a plurality of behavioral time windows; identify a biometric timeseries data object associated with a plurality of biometric time windows; for each biometric time window, determine a desired outcome indicator based at least in part on the biometric timeseries data object; determine a plurality of activity patterns based at least in part on at least one of the behavioral timeseries data object or the biometric timeseries data object, wherein: each activity pattern is identified based at least in part on an occurrence detection time window set comprising at least one of a behavioral occurrence detection time window subset of the plurality of behavioral time windows or a biometric occurrence detection time window subset of the plurality of biometric time windows, and each activity pattern is associated with a biometric impact subset of the plurality of biometric time windows; for each activity pattern, determine an improvement likelihood measure based at least in part on each desired outcome indicator for a biometric time window that is in the biometric impact subset for the activity pattern; generate an activity recommendation machine learning model, wherein the activity recommendation machine learning model maps each activity pattern to the occurrence detection time window set for the activity pattern and the improvement likelihood measure for the activity pattern; and provide access to the activity recommendation machine learning model, wherein the activity recommendation machine learning model is configured to determine, based at least in part on an input behavioral timeseries data object and an input biometric timeseries data object, a recommended activity pattern subset of the plurality of activity patterns.

In accordance with one aspect, a method is provided. In one embodiment, the method comprises identifying a user activity profile for a prediction window, wherein the user activity profile describes one or more recorded user activity events as well as an activity order for the recorded user activity events; identifying a glucose measurement profile for the prediction window, wherein the glucose measurement profile describes one or more recorded glucose measurements associated with the prediction window; generating a glucose measurement time series data object for the prediction window based at least in part on the user activity profile and the glucose measurement profile, wherein the glucose measurement time series data object describes a subset of the one or more glucose measurements that are deemed related to the one or more recorded user activity events and indicates a measurement order for the one or more glucose measurements; processing the glucose measurement time series data object and the user activity profile using a prediction window encoding machine learning model in order to generate an encoded representation for the prediction window; and processing the encoded representation using a metabolic intervention machine learning model in order to determine one or more recommended prediction-based actions for an intervention window subsequent to the prediction window and cause performance of the one or more recommended prediction-based actions.

In accordance with another aspect, a computer program product is provided. The computer program product may comprise at least one computer-readable storage medium having computer-readable program code portions stored therein, the computer-readable program code portions comprising executable portions configured to identify a user activity profile for a prediction window, wherein the user activity profile describes one or more recorded user activity events as well as an activity order for the recorded user activity events; identify a glucose measurement profile for the prediction window, wherein the glucose measurement profile describes one or more recorded glucose measurements associated with the prediction window; generate a glucose measurement time series data object for the prediction window based at least in part on the user activity profile and the glucose measurement profile, wherein the glucose measurement time series data object describes a subset of the one or more glucose measurements that are deemed related to the one or more recorded user activity events and indicates a measurement order for the one or more glucose measurements; process the glucose measurement time series data object and the user activity profile using a prediction window encoding machine learning model in order to generate an encoded representation for the prediction window; and process the encoded representation using a metabolic intervention machine learning model in order to determine one or more recommended prediction-based actions for an intervention window subsequent to the prediction window and cause performance of the one or more recommended prediction-based actions.

In accordance with yet another aspect, an apparatus comprising at least one processor and at least one memory including computer program code is provided. In one embodiment, the at least one memory and the computer program code may be configured to, with the processor, cause the apparatus to identify a user activity profile for a prediction window, wherein the user activity profile describes one or more recorded user activity events as well as an activity order for the recorded user activity events; identify a glucose measurement profile for the prediction window, wherein the glucose measurement profile describes one or more recorded glucose measurements associated with the prediction window; generate a glucose measurement time series data object for the prediction window based at least in part on the user activity profile and the glucose measurement profile, wherein the glucose measurement time series data object describes a subset of the one or more glucose measurements that are deemed related to the one or more recorded user activity events and indicates a measurement order for the one or more glucose measurements; process the glucose measurement time series data object and the user activity profile using a prediction window encoding machine learning model in order to generate an encoded representation for the prediction window; and process the encoded representation using a metabolic intervention machine learning model in order to determine one or more recommended prediction-based actions for an intervention window subsequent to the prediction window and cause performance of the one or more recommended prediction-based actions.

### BRIEF DESCRIPTION OF THE DRAWINGS

Having thus described the invention in general terms, reference will now be made to the accompanying drawings, which are not necessarily drawn to scale, and wherein:

FIG. 1 provides an exemplary overview of a hardware architecture that can be used to practice embodiments of the present invention.

FIG. 2 provides an example predictive data analysis computing entity, in accordance with some embodiments discussed herein.

FIG. 3 provides an example glucose monitoring computing entity, in accordance with some embodiments discussed herein.

FIG. 4 provides an example automated insulin delivery computing entity, in accordance with some embodiments discussed herein.

FIG. 5 provides an example client computing entity, in accordance with some embodiments discussed herein.

FIG. 6 provides an example external computing entity, in accordance with some embodiments discussed herein.

FIG. 7 is a flowchart diagram of an example process for generating predictive metabolic intervention using activity recommendation machine learning models, in accordance with some embodiments discussed herein.

FIG. 8 provides an operational example of a behavioral timeseries data object, in accordance with some embodiments discussed herein.

FIG. 9 provides an operational example of a biometric timeseries data object, in accordance with some embodiments discussed herein.

FIG. 10 is a flowchart diagram of an example process for determining an activity pattern based at least in part on correlations across a behavioral timeseries data object and a biometric timeseries data object, in accordance with some embodiments discussed herein.

FIG. 11 provides an operational example of an occurrence detection time window set, in accordance with some embodiments discussed herein.

FIG. 12 is a flowchart diagram of an example process for performing predictive metabolic intervention using prediction window encoding machine learning models, in accordance with some embodiments discussed herein.

FIG. 13 is a flowchart diagram of an example process for performing predictive metabolic intervention using activity recommendation machine learning models, in accordance with some embodiments discussed herein.

FIG. 14 is a flowchart diagram of an example process for performing predictive metabolic intervention using prediction window encoding machine learning models, in accordance with some embodiments discussed herein.

FIGS. 15A-15F provide operational examples of user activity profiles for various prediction windows, in accordance with some embodiments discussed herein.

FIG. 16 is a flowchart diagram of an example process for generating a glucose measurement timeseries data object for a prediction window, in accordance with some embodiments discussed herein.

FIG. 17 is a data flow diagram of an example process for determining recommended prediction-based actions for an intervention window subsequent to a prediction window, in accordance with some embodiments discussed herein.

### DETAILED DESCRIPTION

Various embodiments of the present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout. Moreover, one of ordinary skill in the art will recognize that the disclosed concepts can be used to perform other types of data analysis.

### I. OVERVIEW AND TECHNICAL ADVANTAGES

Various embodiments of the present invention address technical challenges related to efficiency and effectiveness of performing metabolic predictive data analysis. Some of the efficiency and effectiveness challenges associated with performing metabolic predictive data analysis results from the fact that user activity data (e.g., bolus intake data) and glucose measurement data associated with different predictive windows may be variable in size. This causes challenges for existing machine learning models that expect predictive inputs of a predefined format and structure. Moreover, machine learning models that accept variable-size inputs, such as sequential processing models including recurrent neural networks, are excessively computationally resource-intensive.

Furthermore, various embodiments of the present invention address technical challenges associated with correlating biometric data and behavioral data to perform predictive metabolic intervention by utilizing an activity recommendation machine learning model that maps each activity pattern to the occurrence detection time window set for the activity pattern and the improvement likelihood measure for the activity pattern, where activity patterns may be characterized by event patterns detected based on correlating biometric data and behavioral data, and the improvement likelihood measures may be determined based on biometric impact data. Using the noted techniques, various embodiments of the present invention generate activity recommendation machine learning models using computationally efficient operations configured to temporally align biometric timeseries data and behavioral timeseries data. In doing so, various embodiments of the present invention address technical challenges associated with efficiency and effectiveness of performing metabolic predictive data analysis

In addition, various embodiments of the present invention address technical challenges associated with efficiency and effectiveness of performing metabolic predictive data analysis, and enable performing metabolic predictive data analysis on time windows having diverse user activity profiles, by utilizing a unified machine learning framework that is configured to adapt to variations in the input structures of diverse prediction windows. Accordingly, by reducing the number of machine learning models that should be utilized to perform effective metabolic predictive data analysis in relation to prediction windows having diverse user activity profiles, various embodiments of the present invention both: (i) improve the computational complexity of performing metabolic predictive data analysis by reducing the need for parallel implementation of multiple machine learning models as well as normalizing the outputs of multiple machine learning models, and (ii) reduce the storage costs of performing metabolic predictive data analysis by eliminating the need to store model definition data (e.g., model parameter data and/or model hyper-parameter data) for multiple machine learning models. Accordingly, by addressing the technical challenges associated with efficiency and effectiveness of performing metabolic predictive data analysis, various embodiments of the present invention make substantial technical contributions to improving efficiency and effectiveness of performing metabolic predictive data analysis and to the field of predictive data analysis generally.

Moreover, various embodiments of the present make substantial contributions to the field of treating metabolic dysfunctions. Some of the methods described herein use one or more processors to select a treatment to improve the metabolic health of an individual using glucose readings from an individual obtained after the individual has consumed one or more boluses of known content. The one or more processors may use the glucose readings and a machine learning model to predict a metabolic value. The one or more processors may select the treatment from among a plurality of treatments where the selected treatment is associated with the predicted metabolic value that is closest to an optimal value. By utilizing the noted techniques, various embodiments of the present invention improve treatment of individuals suffering from metabolic dysfunctions.

### II. DEFINITIONS

The term “prediction window” may refer to a data object that describes a period of time whose respective user activity data and glucose measurement data may be used to determine appropriate prediction-based actions to perform during an intervention window subsequent to the prediction window. For example, in some embodiments, a prediction window may describe a particular period of time (e.g., two weeks) prior to a current time, where the user activity data and the physiological measurement data for the noted particular period of time may be used to determine appropriate prediction-based actions to perform during a subsequent period of time after the current time. In some embodiments, the desired length of a period of time described by a prediction window is determined based at least in part on predefined configuration data, where the predefined configuration data may in turn be determined prior to runtime using user-provided data (e.g., system administration data), using rule-based models configured to determine optimal prediction window lengths based at least in part on patient activity data for the prediction window and/or based at least in part on glucose measurement data for the prediction window, using machine learning models configured to determine optimal prediction window lengths, and/or the like. In some embodiments, the desired length of a period of time described by a prediction window is determined based at least in part on configuration data that are dynamically generated at run-time using user-provided data (e.g., system administration data), using rule-based models configured to determine optimal prediction window lengths based at least in part on patient activity data for the prediction window and/or based at least in part on glucose measurement data for the prediction window, using machine learning models configured to determine optimal prediction window lengths, and/or the like. Examples of optimal lengths for periods of times described by prediction windows include twenty-four hours, ten days, two weeks, and/or the like.

For example, in some embodiments, given a prediction window of 24 hours, and given the below schedule, for the purposes of prediction A, Activities B-C and Measurements B-C are deemed relevant, but Activity A and Measurement A are not deemed relevant:

Day 1—8 AM: Activity A

Day 1—8:05 AM: Measurement A

Day 1—10 AM: Activity B

Day 1—10 AM: Measurement B

Day 2—7 AM: Activity C

Day 2—7:05 AM: Measurement C

Day 2—9 AM: Prediction A

The term “recorded user activity event” may refer to a data object that describes attributes (e.g., occurrence, type, magnitude of glucose concentration, magnitude of predicted resulting glucose concentration increase, duration, frequency within a prediction window, and/or the like) of an activity performed by a monitored user, where a corresponding timestamp of the recorded user activity event may be within the period of time described by a corresponding prediction window. Examples of recorded user activity events for a prediction window may include bolus intake events associated with the prediction window, sleep events associated with the prediction window, exercise events associated with the prediction window, drug intake events associated with the prediction window, treatment usage events associated with the prediction window, and/or the like. In some embodiments, a recorded user activity event may describe occurrence of a particular recorded physical user activity and/or occurrence of a particular recorded physical user activity having one or more predefined criteria (e.g., satisfying a calorie consumption threshold).

The term “user activity profile” may refer to a data object that describes recorded user activity events of a corresponding prediction window and indicates an activity order for the noted recorded user activity events. For example, a particular user activity profile may describe that a corresponding prediction window is associated with the following timeline of events: recorded user activity event A1 is performed prior to recorded user activity event A2, which is in turn performed prior to recorded user activity event A3. As another example, another user activity profile may describe that a corresponding prediction window is associated with the following timeline of events: (i) recorded user activity event A1 is performed closely before recorded user activity event A2, which is in turn performed closely before recorded user activity event A3; and (ii) recorded user activity event A4 is performed long after recorded user activity event A3. As yet another example, another user activity profile may describe that a corresponding prediction window is associated with the following timeline of events: (i) recorded user activity event A1 is performed two hours prior to recorded user activity event A2; (ii) recorded user activity event A2 is performed one hour prior to recorded user activity event A3; (iii) recorded user activity event A3 is performed thirty-four minutes prior to recorded user activity event A4; and (iv) recorded user activity event A4 is performed three hours prior to recorded user activity event A5. An example of a user activity profile is a bolus intake profile that describes a sequential occurrence of one or more recorded user activity event. In some embodiments, the user activity profile includes a plurality of recorded user activity events associated with a prediction window that are separated by sufficient time from one another (e.g., separated by at least a length of time that is equal to the amount of time needed for glucose concentration levels of a monitored individual to return to a baseline glucose concentration level).

The term “glucose measurement profile” may refer to a data object that describes one or more recorded glucose concentration measurements (e.g., a portion of the recorded glucose concentration measurements, all of the recorded glucose concentration measurements, and/or the like) for a corresponding prediction window, where each corresponding timestamp for a glucose concentration measurement of the one or more glucose concentration measurements falls within a period of time described by the prediction window. In some embodiments, the timestamp of a glucose concentration measurement is determined based at least in part on a measurement time of the glucose concentration measurement. In some embodiments, a timestamp of a glucose concentration measurement is determined based at least in part on an adjusted measurement time of the glucose concentration measurement, wherein the adjusted measurement time may be determined by adjusting the measurement time of the glucose concentration measurement by a glucose concentration peak interval. In some embodiments, the glucose concentration measurements described by the glucose measurement profile may be determined using continuous glucose monitoring.

The term “glucose measurement timeseries data object” may refer to a data object that describes selected recorded glucose concentration measurements associated with a corresponding prediction window, where the selected recorded glucose concentration measurements are deemed related to (e.g., have timestamps that occur within a predefined time interval subsequent to, such as within 3-5 hours subsequent to) at least one recorded user activity event of a user activity profile. For example, a glucose concentration measurement timeseries data object may describe that a corresponding prediction window is associated with the following timeline of selected glucose concentration measurements: recorded glucose measurement M1 is performed prior to recorded glucose measurement M2, which is in turn performed prior to recorded glucose measurement M3. As another example, another glucose concentration measurement timeseries data object may describe that a corresponding prediction window is associated with the following timeline of selected glucose concentration measurements: (i) recorded glucose measurement M1 is performed closely before recorded glucose measurement M2, which is in turn performed closely before recorded glucose measurement M3; and (ii) recorded glucose measurement M4 is performed long after recorded glucose measurement M4. As yet another example, another glucose concentration measurement timeseries data object may describe that a corresponding prediction window is associated with the following timeline of selected glucose concentration measurements: (i) recorded glucose measurement M1 is performed three hours prior to recorded glucose measurement M2; (i) recorded glucose measurement M2 is performed two hours prior to recorded glucose measurement M3; (iii) recorded glucose measurement M3 is performed thirty-eight minutes prior to recorded glucose measurement M4; and (iv) recorded glucose measurement M4 is performed two hours prior to recorded glucose measurement M5. In some embodiments, the measurement timeseries data object describes the recorded glucose measurements along with one or more extrapolated glucose measurements inferred using one or more temporal extrapolation techniques to fill in the gaps between the noted recorded glucose concentration measurements.

The term “bolus intake event” may refer to a data object that describes a recorded user activity event related to consumption of one or more boluses by a monitored individual. A bolus may be any solid or liquid consumed by the monitored individual. In preferred embodiments, the bolus may be consumed orally—i.e. by eating or drinking the bolus. In some embodiments, the bolus may be injected intravenously. In some embodiments, the bolus may be of known content. Known content need not imply that the exact content of each and every substance in the bolus be known. For example, in some embodiments, only the carbohydrate content of the bolus may be known.

The term “glucose monitoring data” may refer to a data object that describes one or more glucose concentration measurements for a corresponding monitored individual, where each glucose concentration measurement is associated with a corresponding point in time that is associated with the noted glucose concentration measurement. The glucose monitoring data may be calculated using one or more glucose sensors, where the glucose sensors are configured to record glucose concentration measurements and to transmit (e.g., wirelessly, through a wired transmission medium, and/or the like) the recorded glucose concentration measurements to a computing device configured to store glucose concentration measurements. Examples of glucose sensors may include glucose sensors that are in direct contact with at least one of interstitial fluids, blood, other bodily fluids as well as glucose sensors that are not in direct contact with any of the interstitial fluids, blood, other bodily fluids, or tissues, where the latter category may include glucose sensors that use transmission spectroscopy and glucose sensors that use reflection spectroscopy. In some embodiments, the glucose monitoring data is generated by using one or more glucose sensors that collectively enable continuous glucose monitoring for the corresponding monitored individual.

The term “continuous glucose monitoring” may refer to a computer-implemented process that includes recording glucose concentration measurements for a corresponding monitored individual with a continuous frequency and/or with a quasi-continuous frequency, where recording glucose concentration measurements with quasi-continuous frequency may include recording glucose concentration measurements with a frequency deemed sufficiently high to enable measurement of glucose concentrations with an estimated degree of reliability that is deemed to be equivalent to the estimated degree of reliability of measurement of glucose concentrations with continuous frequency. Accordingly, it is important to note that continuous glucose monitoring does not require that readings be instantaneous or absolutely continuous. In some embodiments, continuous glucose monitoring devices provide glucose concentration measurements every five to ten minutes. This frequency may be driven by the need for fidelity of control and by the fact that the most patient-friendly place to sample blood is in the periphery and peripheral blood measurements lag portal measurement, as taking samples over five minutes may reduce the probability that no single abnormal reading will cause incorrect insulin dosing. In some embodiments, in micro-dialysis-based continuous glucose monitoring, sensors may measure glucose in interstitial fluid, where the glucose levels in the interstitial fluid may lag five or more minutes behind blood glucose levels.

The term “continuous glucose monitoring data” may refer to a data object that describes one or more glucose concentration measurements obtained using one or more continuous glucose monitoring processes. In some embodiments, continuous glucose monitoring may performed by one or more continuous glucose monitoring sensors that are configured to record glucose concentration measurements in a continuous manner and/or quasi-continuous manner and to transmit (e.g., wirelessly, through a wired transmission medium, and/or the like) the recorded glucose concentration measurements to a computing device configured to store glucose concentration measurements. Some continuous glucose monitoring sensors use a small, disposable sensor inserted just under the skin. A continuous glucose monitoring sensor may be calibrated with a traditional finger-stick test and the glucose levels in the interstitial fluid may lag five or more minutes behind blood glucose levels. Some continuous glucose monitoring sensors may use non-invasive techniques such as transmission and reflection spectroscopy.

The term “prediction window encoding machine learning model” may refer to a data object that describes parameters and/or hyper-parameters of a machine learning model that is configured to generate a fixed-length representation of a prediction window that integrates the user activity data for the particular prediction window and the glucose measurement data for the particular prediction window. For example, the prediction window encoding machine learning model may be configured to generate a fixed-length representation of a prediction window that integrates the user activity profile for the prediction window and the glucose measurement profile for the prediction window. Examples of prediction window encoding machine learning models include encoder machine learning models, such as autoencoder machine learning models, variational autoencoder machine learning models, encoder machine learning models that include one or more recurrent neural networks such as one or more Long Short Term Memory units, and/or the like. In some embodiments, the prediction window encoding machine learning model may generate a fixed-length representation of a particular prediction window that integrates, in addition to the user activity data for a particular prediction window and the glucose measurement data for a particular prediction window, at least one of the following: (i) a measure of one or more exogenous glucose infusion rates during the prediction window, (ii) a measure of one or more insulin-dependent glucose uptake coefficients during the particular prediction window, (iii) a measure of one or more hepatic glucose production rates during the particular prediction window, (iv) a measure of insulin degradation rates during the particular prediction window, (v) a measure of one or more maximal insulin secretion rates during the particular prediction window, (vi) a measure of one or more insulin-independent glucose uptake rates during the particular prediction window, (vii) a measure of one or more insulin secretion accelerations during the particular prediction window, (viii) a measure of one or more insulin secretion time delays during the particular prediction window, and (ix) a measure of one or more glucose concentration peak intervals during the particular prediction window.

The term “encoded representation” may refer to a data object that describes the fixed-length representation for the particular prediction window that is generated by processing the user activity data for a prediction window and the glucose measurement data for the particular prediction window. In some embodiments, in addition to the user activity data for a particular prediction window and the glucose measurement data for a particular prediction window, the fixed-length representation of a particular prediction window may integrate at least one of the following: (i) a measure of one or more exogenous glucose infusion rates during the prediction window, (ii) a measure of one or more insulin-dependent glucose uptake coefficients during the particular prediction window, (iii) a measure of one or more hepatic glucose production rates during the particular prediction window, (iv) a measure of insulin degradation rates during the particular prediction window, (v) a measure of one or more maximal insulin secretion rates during the particular prediction window, (vi) a measure of one or more insulin-independent glucose uptake rates during the particular prediction window, (vii) a measure of one or more insulin secretion accelerations during the particular prediction window, (viii) a measure of one or more insulin secretion time delays during the particular prediction window, and (ix) a measure of one or more glucose concentration peak intervals during the particular prediction window.

The term “metabolic intervention machine learning model” may refer to a data object that describes parameters and/or hyper-parameters of a machine learning model that is configured to process the encoded representation for a prediction window in order to determine one or more recommended prediction-based actions for an intervention window subsequent to the prediction window. In some embodiments, the metabolic intervention machine learning model is a supervised machine learning model (e.g., a neural network model) trained using labeled data associated with one or more ground-truth prediction windows (e.g., one or more previously-treated prediction windows), where the supervised machine learning model is configured to generate a classification score for each candidate prediction-based action of one or more candidate prediction-based actions and use each classification score for a candidate prediction-based action to determine the recommended prediction-based actions. In some embodiments, the metabolic intervention machine learning model is an unsupervised machine learning model (e.g., a clustering model), where the unsupervised machine learning model is configured to map encoded representation of the prediction window into a multi-dimensional space including mappings of encoded representations of one or more ground-truth prediction windows in order to determine a selected subset of the ground-truth prediction windows whose encoded representation mapping is deemed sufficiently close to the encoded representation mapping of the particular prediction window, and use information about treatment of the selected subset of the ground-truth prediction windows to determine the recommended prediction-based actions.

The term “machine learning model” may refer to a data object that describes parameters, hyper-parameters, defined operations, and/or defined mappings of a model that is configured to process one or more prediction input values (e.g., one or more selected glucose concentration measurements) in accordance with one or more trained parameters of the machine learning models in order to generate a prediction. An example of a machine learning model is a mathematically derived algorithm (MDA). An MDA may comprise any algorithm trained using training data to predict one or more outcome variables. Without limitation, an MDA, as used herein, may comprise machine learning frameworks including neural networks, support vector machines, gradient boosts, Markov models, adaptive Bayesian techniques, and statistical models (e.g., timeseries-based forecast models such as autoregressive models, autoregressive moving average models, and/or an autoregressive integrating moving average models). Additionally and without limitation, an MDA, as used in the singular, may include ensembles using multiple machine learning and/or statistical techniques.

The term “exogenous glucose infusion rate” may refer to a data object that describes the rate at which glucose concentration of a corresponding monitored individual increases following a particular exogenous glucose infusion event, such as at least one of meal ingestion, oral glucose consumption, continuous enteral nutrition, and constant glucose infusion. Exogenous glucose infusion rate may be calculated based at least in part on a model that relates a current exogenous glucose infusion rate to the following: (i) a time parameter describing the current time; (ii) a measure of glucose magnitude following initiation of an activity that leads to exogenous glucose infusion (e.g., consumption of a meal); (iii) glucose distribution volume; and (iv) a glucose concentration peak interval, where (ii)-(iv) may be predefined values. The exogenous glucose infusion rate may be expressed as milligrams per deciliter times inverse of a minute (mg/dl*min⁻¹).

The term “insulin-dependent glucose uptake coefficient” may refer to a data object that describes a coefficient related to the rate at which cells of a corresponding monitored individual utilize glucose in response to receiving insulin at their insulin receptors. Insulin-dependent glucose uptake includes glucose utilization by insulin receptors of muscle cells, fat cells, and other tissue cells, where the noted insulin receptors receive insulin and in response activate a signaling cascade for GLUT4 translocation, which in turn causes the cells to consume the glucose and convert it to energy. As modeled herein, insulin-dependent glucose uptake is the output of a function of both glucose concentrations and insulin concentrations. The insulin-dependent glucose uptake coefficient may take a value that is expressed as the inverse of atomic mass units per milliliters times inverse of a minute ((U/ml*min)⁻¹).

The term “hepatic glucose production rate” may refer to a data object that describes the estimated rate at which liver cells of a corresponding monitored individual produce and secrete insulin in response to production and insulin secretion of glucagon by a-cells in the liver of the corresponding monitored individual, where the noted glucagon production and insulin secretion may exert control over metabolic pathways in the liver in a manner that leads to glucose production. The hepatic glucose production rate may take a value that is described as milligrams per deciliter times inverse of a minute (mg/dl*min⁻¹).

The term “insulin degradation rate” may refer to a data object that describes the estimated rate at which insulin is cleared by insulin-sensitive tissues of a corresponding monitored individual. Insulin clearance activities may be performed by liver, kidney, muscle, adipose cells, and other tissues. The insulin degradation rate may be a factor in an insulin degradation rate function that applies the insulin degradation rate to the insulin concentration. The insulin degradation may take a value that is described as the number of insulin molecules that are degraded by insulin-sensitive tissues in each minute (min⁻¹).

The term “maximal insulin secretion rate” may refer to a data object that describes the estimated maximal rate at which 3-cells in pancreas of a corresponding monitored individual can produce and secrete insulin in response to elevated glucose concentrations in the bloodstream of the corresponding monitored individual. The maximal insulin secretion rate may take a value that is expressed as atomic mass units per milliliter times inverse of a minute (U/ml*min⁻¹).

The term “insulin-independent glucose uptake rate” may refer to a data object that describes the estimated rate at which cells of a corresponding monitored individual utilize glucose, where the noted glucose utilization is performed independent of insulin secretion. Insulin-independent glucose utilization is performed by the brain cells and cells of the nervous system as well as through urination. As modeled herein, insulin-independent glucose utilization is a computational model of glucose concentration. The insulin-independent glucose uptake rate may take a value that is expressed as the number of glucose molecules that are utilized using insulin-independent glucose uptake in each minute (min⁻¹).

The term “half-saturation glucose concentration” may refer to a data object that describes an estimated measure of glucose concentration at a point in time in which half of a maximal degree of possible glucose uptake has been performed for a corresponding monitored individual. The half-saturation glucose concentration can be utilized as a measure of glucose uptake capability of a monitored individual. The half-saturation glucose concentration can take a value that is expressed as milligrams per deciliter (mg/dl).

The term “insulin secretion acceleration” may refer to a data object that describes an estimated measure of the rate at which 3-cells of pancreas of a corresponding monitored individual accelerate insulin production and insulin secretion when the noted p-cells detect heightened levels of glucose concentration in the bloodstream of the corresponding monitored individual. The insulin secretion acceleration may take the form of an exponential parameter, such as the Hill coefficient of a Hill function configured to model the glucose-insulin regulatory system.

The term “insulin secretion time delay” may refer to a data object that describes an estimated measure of temporal delay between appearance of heightened glucose concentrations in the bloodstream of a corresponding monitored individual and a time associated with insulin secretion by 3-cells of the pancreas. For example, the insulin secretion time delay may describe the estimated measure of temporal delay between appearance of heightened glucose concentrations in the bloodstream of the corresponding monitored individual and a time associated with initiation of insulin secretion by 3-cells of the pancreas. As another example, the insulin secretion time delay may describe the estimated measure of temporal delay between appearance of heightened glucose concentrations in the bloodstream of the corresponding monitored individual and a time associated with termination of insulin secretion by 3-cells of the pancreas.

The term “glucose concentration peak interval” may refer to a data object that describes an estimated length of a time between the first appearance of the glucose in the bloodstream of a corresponding monitored individual as a result of a exogenous glucose infusion and peak of glucose in the blood stream of the corresponding monitored individual as a result of the exogenous glucose infusion. For example, the glucose concentration peak interval may describe a time delay between first appearance of exogenously-infused glucose in the bloodstream of the monitored individual as a result of a meal ingestion and a peak of meal absorption. The glucose concentration peak interval may take a value that is expressed as minutes (min).

The term “behavioral timeseries data object” may refer to a data construct that is configured to describe a recorded behavioral activity description measure for a monitored individual over a plurality of time periods. For example, in some embodiments, the behavioral timeseries data object may describe a recorded movement velocity of a monitored individual over a plurality of time windows. As another example, in some embodiments, the behavioral timeseries data object may describe a recorded calorie consumption rate of a monitored individual over a plurality of time windows. As yet another example, in some embodiments, the behavioral timeseries data object may describe a recorded pulse rate of a monitored individual over a plurality of time windows. As a further example, in some embodiments, the behavioral timeseries data object may describe a recorded bodily exercise frequency of a monitored individual over a plurality of time windows. In some embodiments, the data described by the behavioral timeseries data object is determined by using one or more behavioral sensor devices that are configured to monitor behavioral conditions of the monitored individual periodically or continuously over time and report the noted behavioral conditions to one or more server computing entities, where the server computing entities are configured to generate the behavioral timeseries data object based at least in part on the behavioral condition data that is received from the noted one or more behavioral sensors. In some embodiments, the behavioral timeseries data object is generated based at least in part on each plurality of recorded observations for an individual of a plurality of individuals, and each plurality of recorded observations for an individual is determined based at least in part on a plurality of observation time windows for the individual, and the plurality of behavioral time windows comprise each plurality of observation time windows for an individual.

The term “behavioral time window” may refer to a data construct that is configured to describe a time period of the plurality of time periods across which a behavioral timeseries data object is calculated. For example, in some embodiments, a plurality of behavioral time windows describes a plurality of defined time periods that follow each other in a continuous manner across which a behavioral timeseries data object is calculated. As another example, in some embodiments, a plurality of behavioral time windows describes a plurality of disjoint time periods across which a behavioral timeseries data object is calculated. As yet another example, in some embodiments, a plurality of behavioral time windows describes: (i) one or more sets of continuous time periods, where each set describes a plurality of defined time periods that follow each other in a continuous manner across which a behavioral timeseries data object is calculated, and (ii) one or more sets of disjoint time periods, where each set describes a plurality of disjoint time periods across which a behavioral timeseries data object is calculated.

The term “biometric timeseries data object” may refer to a data construct that is configured to describe a recorded biometric measure for a monitored individual over a plurality of time periods. For example, in some embodiments, the biometric timeseries data object may describe a recorded blood glucose level of a monitored individual over a plurality of time windows. As another example, in some embodiments, the biometric timeseries data object may describe a recorded heart rate of a monitored individual over a plurality of time windows. As yet another example, in some embodiments, the biometric timeseries data object may describe a recorded pulse rate of a monitored individual over a plurality of time windows. As a further example, in some embodiments, the biometric timeseries data object may describe a recorded bodily temperature of a monitored individual over a plurality of time windows. As an additional example, in some embodiments, the biometric timeseries data object may describe a recorded breathing rate of a monitored individual over a plurality of time windows. In some embodiments, the data described by the biometric timeseries data object is determined by using one or more biometric sensor devices that are configured to monitor biometric conditions of the monitored individual periodically or continuously over time and report the noted biometric conditions to one or more server computing entities, where the server computing entities are configured to generate the biometric timeseries data object based at least in part on the biometric condition data that is received from the noted one or more biometric sensors. In some embodiments, the biometric timeseries data object is generated based at least in part on one or more recorded longitudinal observations of a corresponding individual across the plurality of biometric time windows. In some embodiments, the biometric timeseries data object is generated based at least in part on each plurality of recorded observations for an individual of a plurality of individuals, each plurality of recorded observations for an individual is determined based at least in part on a plurality of observation time windows for the individual, and the plurality of biometric time windows comprise each plurality of observation time windows for an individual.

The term “biometric time window” may refer to a data construct that is configured to describe a time period of the plurality of time periods across which a biometric timeseries data object is calculated. For example, in some embodiments, a plurality of biometric time windows describes a plurality of defined time periods that follow each other in a continuous manner across which a biometric timeseries data object is calculated. As another example, in some embodiments, a plurality of biometric time windows describes a plurality of disjoint time periods across which a biometric timeseries data object is calculated. As yet another example, in some embodiments, a plurality of biometric time windows describes: (i) one or more sets of continuous time periods, where each set describes a plurality of defined time periods that follow each other in a continuous manner across which a biometric timeseries data object is calculated, and (ii) one or more sets of disjoint time periods, where each set describes a plurality of disjoint time periods across which a biometric timeseries data object is calculated.

The term “desired outcome indicator” may refer to a data construct that is configured to describe if a time window is associated with a biometric condition that is deemed to be a target biometric condition that a predictive data analysis framework is configured to detect. For example, the desired outcome indicator for a time window may be determined based at least in part on whether a biometric measure for the time window has a value that falls within a threshold range for the biometric measure. As another example, the desired outcome indicator for a time window may be determined based at least in part on whether the time-in-range of the blood glucose level for the time window satisfies a threshold time-in-range condition, where the time-in-range of the blood glucose level for a time window may describe a ratio of the time that the blood glucose level for the time window is within a target range (e.g., a target range deemed to indicate abnormal and/or critical blood glucose level). In some embodiments, a predictive data analysis computing entity determines a desired outcome indicator for each biometric time window based at least in part on whether the biometric measure described for the biometric time window by a biometric timeseries data object falls within a threshold range for the biometric measure. For example, the predictive data analysis computing entity may determine a desired outcome indicator for each biometric time window based at least in part on whether the blood glucose level for the biometric time window by a biometric timeseries data object falls within a threshold range for the blood glucose level. As another example, the predictive data analysis computing entity may determine a desired outcome indicator for each biometric time window based at least in part on whether the recorded heartrate for the biometric time window by a biometric timeseries data object falls within a threshold range for the recorded heartrate. As yet another example, the predictive data analysis computing entity may determine a desired outcome indicator for each biometric time window based at least in part on whether the recorded breathing rate for the biometric time window by a biometric timeseries data object falls within a threshold range for the recorded breathing rate. In some embodiments, each desired outcome indicator for a biometric time window is a target time in range measure for the corresponding biometric time window.

The term “occurrence detection time window subset” may refer to a data construct that is configured to describe a plurality of time windows that are deemed to describe an activity pattern. In some embodiments, the occurrence detection window subset may include a plurality of behavioral time windows that are deemed to describe an activity pattern. For example, the occurrence detection time window subset may include a plurality of behavioral time windows that are deemed to describe an activity pattern as determined based at least in part on behavioral description measures associated with the behavioral time window. In some embodiments, the occurrence detection window subset may include a plurality of biometric time windows that are deemed to describe an activity pattern. For example, the occurrence detection time window subset may include a plurality of biometric time windows that are deemed to describe an activity pattern as determined based at least in part on biometric measures associated with the plurality of biometric time windows. For example, the occurrence detection window subset may include a plurality of behavioral time windows and a plurality of biometric time windows, where correlating the behavioral description measures of the plurality of behavioral time windows and the biometric measures of the plurality of biometric time windows indicates that the plurality of behavioral time windows and the plurality of biometric time windows collectively describe a detected/identified activity. In some embodiments, if an activity pattern is detected solely based at least in part on behavioral data (e.g., based at least in part on behavioral timeseries data objects), then the occurrence detection window set for that activity pattern includes any time periods in the behavioral data that are used to detect an activity pattern. In some embodiments, if an activity pattern is detected solely based at least in part on biometric data (e.g., based at least in part on biometric timeseries data objects), then the occurrence detection window set for that activity pattern includes any time periods in the biometric data that are used to detect an activity pattern. In some embodiments, if an activity pattern is detected based at least in part on both behavioral data and biometric data (e.g., based at least in part on behavioral timeseries data objects and biometric timeseries data objects), then the occurrence detection window set for that activity pattern includes any time periods in the behavioral data and any time periods in the biometric data that are used to detect the activity pattern, where the time periods in the behavioral data and the time periods in the biometric data are deemed to be temporally correlated in a manner that are deemed to refer to the same activity pattern.

The term “behavioral occurrence detection time window subset” may refer to a data construct that is configured to describe a plurality of behavioral time windows that are deemed to describe an activity pattern. In some embodiments, the occurrence detection window subset may include a plurality of behavioral time windows that are deemed to describe an activity pattern. For example, the occurrence detection time window subset may include a plurality of behavioral time windows that are deemed to describe an activity pattern as determined based at least in part on behavioral description measures associated with the behavioral time window. In some embodiments, if an activity pattern is detected solely based at least in part on behavioral data (e.g., based at least in part on behavioral timeseries data objects), then the occurrence detection window set for that activity pattern includes any time periods in the behavioral data that are used to detect an activity pattern. Examples of behavioral occurrence detection time window subsets include sets of behavioral time windows that describe intensive physical activity patterns, intense exercise activity patterns, substantial food intake activity patterns, fasting activity patterns, and/or the like. For example, in some embodiments, a plurality of behavioral time windows may be in a behavioral occurrence detection time window subset if monitored behavioral activity measures (e.g., movement velocity measures, heart rate measures, and/or the like) for the plurality of behavioral time windows (e.g., as described by a behavioral timeseries data object) describe that a monitored individual has engaged in a desired/target activity pattern (e.g., running, exercise, and/or the like).

The term “biometric occurrence detection time window subset” may refer to a data construct that is configured to describe a plurality of biometric time windows that are deemed to describe an activity pattern. In some embodiments, the occurrence detection window subset may include a plurality of biometric time windows that are deemed to describe an activity pattern. For example, the occurrence detection time window subset may include a plurality of biometric time windows that are deemed to describe an activity pattern as determined based at least in part on biometric measures associated with the plurality of biometric time windows. In some embodiments, if an activity pattern is detected solely based at least in part on behavioral data (e.g., based at least in part on behavioral timeseries data objects), then the occurrence detection window set for that activity pattern includes any time periods in the behavioral data that are used to detect an activity pattern. In some embodiments, if an activity pattern is detected solely based at least in part on biometric data (e.g., based at least in part on biometric timeseries data objects), then the occurrence detection window set for that activity pattern includes any time periods in the biometric data that are used to detect an activity pattern. Examples of biometric occurrence detection time window subsets include sets of biometric time windows that describe intensive physical activity patterns, intense exercise activity patterns, substantial food intake activity patterns, fasting activity patterns, and/or the like. For example, in some embodiments, a plurality of biometric time windows may be in a biometric occurrence detection time window subset if monitored biometric measures (e.g., glucose levels, heart rates, breathing rates, etc.) associated with the plurality of biometric time windows (e.g., as described by a biometric timeseries data object) describe that a monitored individual has engaged in a desired/target activity pattern (e.g., calorie intake, running, exercise, and/or the like). As another example, in some embodiments, a plurality of biometric time windows may be in a biometric occurrence detection time window subset if glucose levels associated with the plurality of biometric time windows describe that a monitored individual has performed a calorie intake. As yet another example, in some embodiments, a plurality of biometric time windows may be in a biometric occurrence detection time window subset if breathing rates and/or heart rates associated with the plurality of biometric time windows describe that a monitored individual has engaged in intense physical activity. As an additional example, in some embodiments, a plurality of biometric time windows may be in a biometric occurrence detection time window subset if breathing rates and/or heart rates associated with the plurality of biometric time windows describe that a monitored individual has engaged in high-stress activity.

The term “biometric impact subset” may refer to a data construct that is configured to describe a plurality of time windows that describe biometric impact data describing biometric impacts of an activity pattern. In some embodiments, while the occurrence detection time window subset includes a plurality of time windows that are deemed to describe occurrence of an activity pattern, the biometric impact subset of the activity pattern includes a plurality of biometric time windows that are deemed to describe biometric impacts of an activity pattern. For example, if the occurrence detection time window subset for an activity pattern includes time windows t₁-t₄, and if the biometric impact subset for the activity pattern is deemed to begin n time windows after the termination of the occurrence detection time window subset and last for m time windows, then the biometric impact subset for the activity pattern may include the time windows t₄₊ₙ tot₄₊ₙ₊ₘ. In some embodiments, in the described example, at least one of n and m may be determined (e.g., based at least in part on historical activity monitoring data) in accordance with an activity pattern type of the corresponding activity pattern. In some embodiments, each activity pattern is associated with a plurality of time windows in the biometric data where a proposed system can see the impact of the activity pattern in terms of the desired outcome variable. In some of the noted embodiments, this plurality of time windows in the glucose data is referred to as the biometric impact subset for the activity pattern.

The term “improvement likelihood measure” may refer to a data construct that is configured to describe a measure of the likelihood that occurrence of an activity pattern is likely to cause a biometric condition that is deemed to be a target biometric condition that a predictive data analysis framework is configured to detect. In some embodiments, the improvement likelihood measure for an activity pattern is determined based at least in part on the biometric impact subset for the activity pattern, e.g., based at least in part on whether the desired outcome indicators for at least n (e.g., at least one) biometric time windows in the biometric impact subset for the activity pattern describe that the biometric time window is associated with a biometric condition that is deemed to be a target biometric condition that a predictive data analysis framework is configured to detect, or based at least in part on how many desired outcome indicators for biometric time windows in the biometric impact subset for the activity pattern describe that the biometric time window is associated with a biometric condition that is deemed to be a target biometric condition that a predictive data analysis framework is configured to detect. For example, an activity pattern may be associated with an improvement likelihood measure that describes how many of the biometric time windows in the biometric impact subset for the activity pattern are associated with a corresponding desired outcome indicator that describes that the biometric time window is likely to cause a biometric condition that is deemed to be a target biometric condition that a predictive data analysis framework is configured to detect. In some embodiments, if an activity pattern is associated with a biometric impact subset including n biometric time windows, where m of the n biometric time windows are deemed likely to cause a biometric condition that is deemed to be a target biometric condition that a predictive data analysis framework is configured to detect, and n−m of the biometric time windows are deemed unlikely to cause a biometric condition that is deemed to be a target biometric condition that a predictive data analysis framework is configured to detect, then the improvement likelihood measure for the activity pattern is m. In some embodiments, if an activity pattern is associated with a biometric impact subset including n biometric time windows, where m of the n biometric time windows are deemed likely to cause a biometric condition that is deemed to be a target biometric condition that a predictive data analysis framework is configured to detect, and n−m of the biometric time windows are deemed unlikely to cause a biometric condition that is deemed to be a target biometric condition that a predictive data analysis framework is configured to detect, then the improvement likelihood measure for the activity pattern is m n. In some embodiments, if an activity pattern is associated with a biometric impact subset including n biometric time windows, where m of the n biometric time windows are deemed likely to cause a biometric condition that is deemed to be a target biometric condition that a predictive data analysis framework is configured to detect, and n−m of the biometric time windows are deemed unlikely to cause a biometric condition that is deemed to be a target biometric condition that a predictive data analysis framework is configured to detect, then the improvement likelihood measure for the activity pattern is (n−m) n.

The term “activity pattern” may refer to a data construct that describes a designation that may be associated with an occurrence detection time window set based at least in part on at least one of the following: (i) detected patterns in behavioral timeseries data objects, (ii) detected patterns in biometric timeseries data objects, and (iii) detected patterns in correlation data inferred by correlating one or more behavioral timeseries data objects and one or more biometric timeseries data objects. Examples of activity patterns include designations that describe performing intense physical activities, performing calorie intake activities, performing physical exercise activities, and/or the like. In some embodiments, the activity patterns include one or more of the following: (i) biometric activity patterns that are determined solely based at least in part on detected patterns in biometric timeseries data objects (e.g., such that the occurrence detection time window set for each biometric activity pattern comprises the biometric occurrence detection time window subset for the biometric activity pattern), (ii) behavioral activity patterns that are determined solely based at least in part on detected patterns in behavioral timeseries data objects (e.g., such that the occurrence detection time window set for each behavioral activity pattern comprises the behavioral occurrence detection time window subset for the behavioral activity pattern), and (iii) behavioral-biometric activity patterns that are determined based at least in part on detected patterns in correlation data inferred by correlating one or more behavioral timeseries data objects and one or more biometric timeseries data objects (e.g., such that the occurrence detection time window set for each behavioral-biometric activity pattern comprises both the behavioral occurrence detection time window subset for the behavioral-biometric activity pattern and the biometric occurrence detection time window subset for the behavioral-biometric activity pattern, and each behavioral-biometric activity pattern is determined based at least in part on one or more detected cross-timeseries correlations across the plurality of behavioral time windows and the plurality of biometric time windows).

The term “activity recommendation machine learning model” may refer to a data construct that is configured to associate each activity pattern of a plurality of activity patterns to at least one of the following: (i) an occurrence detection time window set for the activity pattern, and (ii) an improvement likelihood measure for the activity pattern. In some embodiments, the activity recommendation machine learning model maps each activity pattern to the occurrence detection time window set for the activity pattern and the improvement likelihood measure for the activity pattern. In some embodiments, by using an activity recommendation machine learning model, a predictive data analysis computing entity can: (i) process an input behavioral timeseries data object for a monitored individual and/or an input biometric timeseries data object for a monitored individual in order to determine one or more activity patterns in the noted input data objects based at least in part on at least one of the input behavioral timeseries data object, the input biometric timeseries data object, and correlating the input biometric timeseries data object and the input behavioral timeseries data object, (ii) determine the improvement likelihood measures for the activity patterns in the noted input data objects to select a selected subset of the noted activity patterns (e.g., to select the top n activity patterns having the top n improvement likelihood measures, to select the activity patterns whose improvement likelihood measures satisfy an improvement likelihood measure, and/or the like), and (iii) present the selected subset of the noted activity patterns to an end user of a predictive data analysis computing entity. In some embodiments, mappings between activity patterns and occurrence detection time window sets as described by the activity recommendation machine learning model can be used to infer activity patterns based at least in part on input behavioral timeseries data objects and input biometric timeseries data objects. In some embodiments, mappings between activity patterns and improvement likelihood measures can be used to select a selected subset of inferred detectivity patterns, where the inferred activity patterns may be inferred based at least in part on input behavioral timeseries data objects and input biometric timeseries data objects in accordance with mappings between activity patterns and occurrence detection time window sets. In some embodiments, a predictive data analysis computing entity is configured to provide access to the activity recommendation machine learning model, wherein the activity recommendation machine learning model is configured to determine, based at least in part on an input behavioral timeseries data object and an input biometric timeseries data object, a recommended activity pattern subset of the plurality of activity patterns.

### III. COMPUTER PROGRAM PRODUCTS, METHODS, AND COMPUTING ENTITIES

Embodiments of the present invention may be implemented in various ways, including as computer program products that comprise articles of manufacture. Such computer program products may comprise one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may comprise a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media comprise all computer-readable media (including volatile and non-volatile media).

In one embodiment, a non-volatile computer-readable storage medium may comprise a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also comprise a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also comprise read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also comprise conductive-bridging random access memory (CBRAM), phase-change random access memory (PRAM), ferroelectric random-access memory (FeRAM), non-volatile random-access memory (NVRAM), magneto-resistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may comprise random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations.

Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### IV. EXEMPLARY SYSTEM ARCHITECTURE

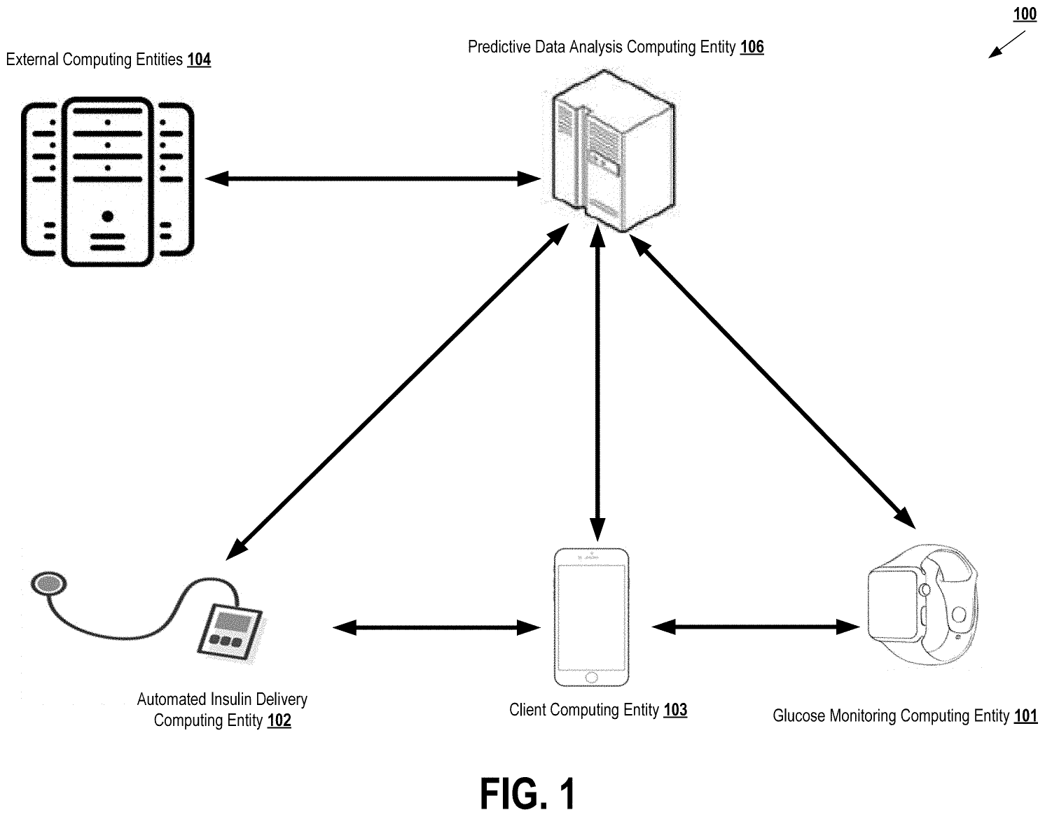

FIG. 1 depicts an architecture 100 for performing predictive metabolic intervention. The architecture 100 includes a predictive data analysis computing entity 106, a glucose monitoring computing entity 101, an automated insulin delivery computing entity 102, a client computing entity 103, and one or more external computing entities 104. Communication between the noted computing entities may be facilitated using one or more communication networks. Examples of communication networks comprise any wired or wireless communication network including, for example, a wired or wireless local area network (LAN), personal area network (PAN), metropolitan area network (MAN), wide area network (WAN), short-range communication networks (e.g., Bluetooth networks), or the like, as well as any hardware, software and/or firmware required to implement it (such as, e.g., network routers, and/or the like).

The predictive data analysis computing entity 106 may be configured to receive glucose monitoring data (e.g., continuous glucose monitoring data) from the glucose monitoring computing entity 101, process the glucose monitoring data to determine one or more prediction-based actions, and perform the one or more prediction-based actions by interacting with at least one of the glucose monitoring computing entity 101, the automated insulin delivery computing entity 102, and the external computing entities 104.

For example, the predictive data analysis computing entity 106 may communicate glucose-insulin predictions generated based at least in part on the glucose monitoring data to the glucose monitoring computing entity 101 and/or to the external computing entities 104. As another example, the predictive data analysis computing entity 106 may, in response to determining a positive insulin need determination based at least in part on the glucose monitoring data for a monitored individual, communicate one or more insulin delivery instructions to the automated insulin delivery computing entity 102 that is associated with the monitored individual. In some embodiments, some or all of the functions of the predictive data analysis computing entity 106 are performed by the glucose monitoring computing entity 101. In some of the noted embodiments, the glucose monitoring computing entity 101 is configured to receive glucose monitoring data (e.g., continuous glucose monitoring data) from the glucose monitoring computing entity 101, process the glucose monitoring data to determine one or more prediction-based actions, and perform the one or more prediction-based actions by interacting with at least one of the glucose monitoring computing entity 101, the automated insulin delivery computing entity 102, and the external computing entities 104.

The glucose monitoring computing entity 101 may be configured to record glucose concentration measurements for a monitored individual and to communicate the glucose concentration measurements to at least one of the predictive data analysis computing entity 106, the glucose monitoring computing entity 101, and the external computing entities 104. In some embodiments, the glucose monitoring computing entity 101 is directly connected to the predictive data analysis computing entity 106. In some embodiments, the glucose monitoring computing entities 101 is configured to transmit the glucose concentration measurements to the glucose monitoring computing entity 101, and the glucose monitoring computing entity 101 is configured to forward the glucose concentration measurements received from the glucose monitoring computing entity 101 to the predictive data analysis computing entity 106.

In some embodiments, the glucose monitoring computing entity 101 includes one or more continuous glucose monitoring sensors. Some continuous glucose monitoring sensors use a small, disposable sensor inserted just under the skin. The sensor must be calibrated with a traditional finger-stick test and the glucose levels in the interstitial fluid may lag five or more minutes behind blood glucose levels. Other continuous glucose monitoring sensors may use non-invasive techniques such as transmission and reflection spectroscopy. In some embodiments, the glucose monitoring computing entity 101 includes a display device that is configured to display a user interface. Such a user interface could include, for example, one or more of a display screen, an audio speaker, or a tactile output. In some embodiments, the user interface allows the user to communicate with the system. For example, in some embodiments, the system may include a keyboard, microphone, or touch screen allowing the user to enter information related to glucose levels such as the type, time, and amount of food consumed or the type, time, intensity of physical activity, medicines used and in what amount, stress level, depression level, energy level, location, or an environmental condition.

The automated insulin delivery computing entity 102 may be configured to receive insulin delivery instructions from the predictive data analysis computing entity 106 and to perform the received insulin delivery instructions by ingesting insulin to the bloodstream of a monitored individual in amounts specified by the insulin delivery instructions. In some embodiments, the automated insulin delivery computing entity 102 includes one or more insulin pumps, where an insulin pump is a computerized device that is configured to mimic the operation of the pancreas by secreting insulin amounts, as well as tubing mechanisms and an infusion set. In some embodiments, the automated insulin delivery computing entity 102 directly receives insulin delivery instructions from the predictive data analysis computing entity 106. In some embodiments, the predictive data analysis computing entity 106 transmits the insulin delivery instructions to the glucose monitoring computing entity 101, and the glucose monitoring computing entity 101 in turn forwards the insulin delivery instructions to the automated insulin delivery computing entity 102. In some embodiments, the automated insulin delivery computing entity 102 includes a display device that is configured to display a user interface. Such a user interface could include, for example, one or more of: a display screen, an audio speaker, or a tactile output. In some embodiments, the user interface allows the user to communicate with the system. For example, in some embodiments, the system may include a keyboard, microphone, or touch screen allowing the user to enter information related to glucose levels such as the type, time, and amount of food consumed or the type, time, intensity of physical activity, medicines used and in what amount, stress level, depression level, energy level, location, or an environmental condition.

The client computing entity 103 may be configured to enable user display of glucose monitoring data and/or user configuration of predictive management actions performed by the predictive data analysis computing entity 106. Examples of client computing entities 103 include smartphone devices, tablet devices, personal computer devices, and/or the like. The client computing entity 103 may include a short-range communication network receiver (e.g., a Bluetooth receiver) that is configured to receive glucose monitoring data from the glucose monitoring computing entity 101 and/or to provide insulin delivery instructions to the automated insulin delivery computing entity 102. The client computing entity 103 may further be configured to provide glucose monitoring data received from the glucose monitoring computing entity 101 to the predictive data analysis computing entity 106 and/or to receive insulin delivery instructions from the predictive data analysis computing entity 106 before transmission of the noted insulin delivery instructions to the automated insulin delivery computing entity 102.

In some embodiments, the glucose monitoring computing entity 101 is configured to perform some or all of the functionalities of the predictive data analysis computing entity 106. In some of the noted embodiments, the glucose monitoring computing entity 101 is configured to receive glucose monitoring data (e.g., continuous glucose monitoring data) from the glucose monitoring computing entity 101, process the glucose monitoring data to determine one or more prediction-based actions, and perform the one or more prediction-based actions by interacting with at least one of the glucose monitoring computing entity 101, the automated insulin delivery computing entity 102, and the external computing entities 104.

The external computing entities 104 may be configured to receive notification data and/or user interface data generated by the predictive data analysis computing entity 106 and perform corresponding actions based at least in part on the receive data. For example, an external computing entity 104 may be configured to generate one or more physician alerts and/or one or more healthcare provider alerts based at least in part on the notification data provided by the predictive data analysis computing entity 106. As another example, an external computing entity 104 may be configured to generate one or more automated physician appointments, automated medical notes, automated prescription recommendations, and/or the like based at least in part on the notification data received from the predictive data analysis computing entity 106. As yet another example, an external computing entity 104 may be configured to enable an end-user device associated with the external computing entity 104 to display a user interface, where the user interface may have been generated based at least in part on the user interface data provided by the predictive data analysis computing entity 106. Examples of external computing entities 104 include hospital servers, physician servers, laboratory servers, emergency room servers, urgent care centers, research institution servers, and/or the like.

### Exemplary Predictive Data Analysis Computing Entity

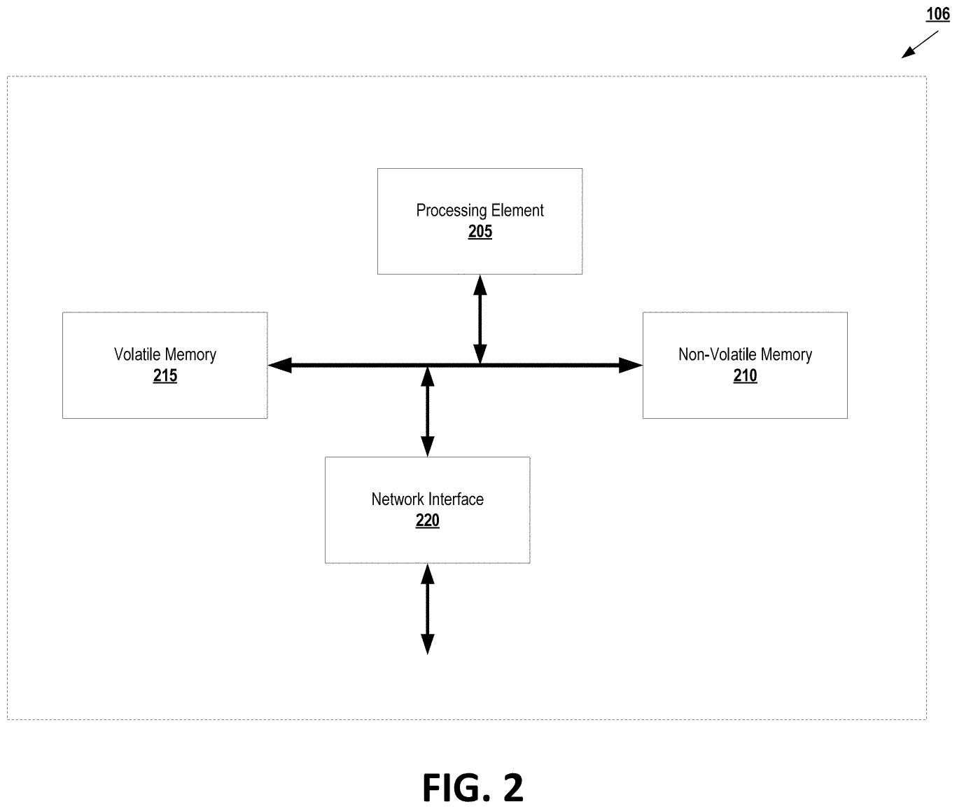

FIG. 2 provides a schematic of a predictive data analysis computing entity 106 according to one embodiment of the present invention. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the predictive data analysis computing entity 106 may also comprise one or more network interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the predictive data analysis computing entity 106 may comprise or be in communication with one or more processing elements 205 (also referred to as processors, processing circuitry, and/or similar terms used herein interchangeably) that communicate with other elements within the predictive data analysis computing entity 106 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, another circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the predictive data analysis computing entity 106 may further comprise or be in communication with non-volatile media (also referred to as non-volatile storage, memory, memory storage, memory circuitry and/or similar terms used herein interchangeably). In one embodiment, the non-volatile storage or memory may comprise one or more non-volatile storage or memory media 210, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or information/data that is stored in a computer-readable storage medium using one or more database models, such as a hierarchical database model, network model, relational model, entity-relationship model, object model, document model, semantic model, graph model, and/or the like.

In one embodiment, the predictive data analysis computing entity 106 may further comprise or be in communication with volatile media (also referred to as volatile storage, memory, memory storage, memory circuitry and/or similar terms used herein interchangeably). In one embodiment, the volatile storage or memory may also comprise one or more volatile storage or memory media 215, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the predictive data analysis computing entity 106 with the assistance of the processing element 205 and operating system.

As indicated, in one embodiment, the predictive data analysis computing entity 106 may also comprise one or more network interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive data analysis computing entity 106 may be configured to communicate via wireless client communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the predictive data analysis computing entity 106 may comprise or be in communication with one or more input elements, such as a keyboard input, a mouse input, a touch screen/display input, motion input, movement input, audio input, pointing device input, joystick input, keypad input, and/or the like. The predictive data analysis computing entity 106 may also comprise or be in communication with one or more output elements (not shown), such as audio output, video output, screen/display output, motion output, movement output, and/or the like.

### Exemplary Glucose Monitoring Computing Entity

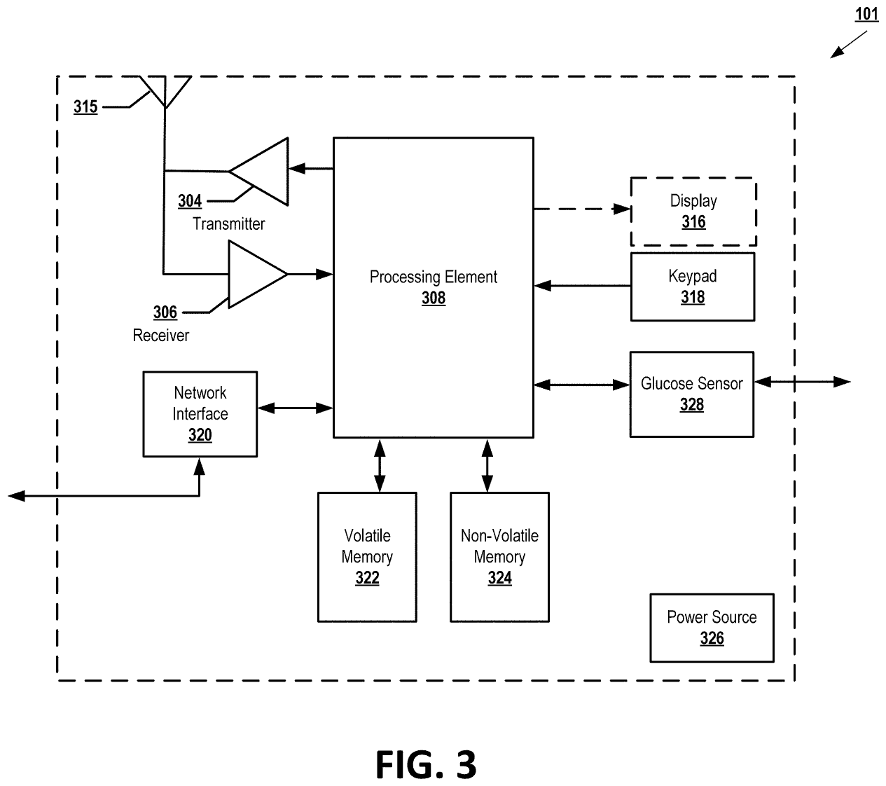

FIG. 3 provides an illustrative schematic representative of a glucose monitoring computing entity 101 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Glucose monitoring computing entities 101 can be operated by various parties. As shown in FIG. 3, the glucose monitoring computing entity 101 can comprise an antenna 312, a transmitter 304 (e.g., radio), a receiver 306 (e.g., radio), a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly, a power source 326, and a glucose sensor 328.

The signals provided to and received from the transmitter 304 and the receiver 306, correspondingly, may comprise signaling information/data in accordance with air interface standards of applicable wireless systems. In this regard, the glucose monitoring computing entity 101 may be capable of operating with one or more air interface standards, communication protocols, modulation types, and access types. More particularly, the glucose monitoring computing entity 101 may operate in accordance with any of a number of wireless communication standards and protocols, such as those described above with regard to the predictive data analysis computing entity 106. In a particular embodiment, the glucose monitoring computing entity 101 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the glucose monitoring computing entity 101 may operate in accordance with multiple wired communication standards and protocols, such as those described above with regard to the predictive data analysis computing entity 106 via a network interface 320.

Via these communication standards and protocols, the glucose monitoring computing entity 101 can communicate with various other entities using concepts such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The glucose monitoring computing entity 101 can also download changes, add-ons, and updates, for instance, to its firmware, software (e.g., including executable instructions, applications, program modules), and operating system.

According to one embodiment, the glucose monitoring computing entity 101 may comprise location determining aspects, devices, modules, functionalities, and/or similar words used herein interchangeably. For example, the glucose monitoring computing entity 101 may comprise outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This information/data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the glucose monitoring computing entity's 102 position in connection with a variety of other systems, including cellular towers, Wi-Fi access points, and/or the like. Similarly, the glucose monitoring computing entity 101 may comprise indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may comprise the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

In some embodiments, the transmitter 304 may include one or more Bluetooth transmitters. In some embodiments, the receiver 306 may include one or more Bluetooth receivers. The Bluetooth transmitters and/or the Bluetooth receivers may be configured to communicate with at least one of the client computing entity 103 and the predictive data analysis computing entity 106. In some embodiments, the transmitter 304 may include one or more WAN transmitters. In some embodiments, the receiver 306 may include one or more WAN receivers. The WAN transmitters and/or the WAN receivers may be configured to communicate with at least one of the client computing entity 103 and the predictive data analysis computing entity 106.

The power source 326 may include electric circuitry configured to enable powering the glucose monitoring computing entity 101. The power source 326 may include one or more batteries, such as a rechargeable lithium-ion (Li-Ion) battery, that are configured to act as sources of electric power for the glucose monitoring computing entity 101.

The glucose monitoring computing entity 101 may also comprise a user interface (that can optionally comprise a display 316 coupled to a processing element 308) and/or a user input interface (coupled to a processing element 308). For example, the user interface may be a user application, browser, user interface, and/or similar words used herein interchangeably executing on and/or accessible via the glucose monitoring computing entity 101 to interact with and/or cause display of information/data from the predictive data analysis computing entity 106, as described herein. The user input interface can comprise any of a number of devices or interfaces allowing the glucose monitoring computing entity 101 to receive data, such as a keypad 318 (hard or soft), a touch display, voice/speech or motion interfaces, or other input device. In embodiments including a keypad 318, the keypad 318 can comprise (or cause display of) the conventional numeric (0-9) and related keys (#, *), and other keys used for operating the glucose monitoring computing entity 101 and may comprise a full plurality of alphabetic keys or plurality of keys that may be activated to provide a full plurality of alphanumeric keys. In addition to providing input, the user input interface can be used, for example, to activate or deactivate certain functions, such as screen savers and/or sleep modes.

The glucose monitoring computing entity 101 can also comprise volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the glucose monitoring computing entity 101. As indicated, this may comprise a user application that is resident on the entity or accessible through a browser or other user interface for communicating with the predictive data analysis computing entity 106 and/or various other computing entities.

In another embodiment, the glucose monitoring computing entity 101 may comprise one or more components or functionalities that are the same or similar to those of the predictive data analysis computing entity 106, as described in greater detail above. As will be recognized, these architectures and descriptions are provided for exemplary purposes only and are not limiting to the various embodiments.

### Exemplary Automated Insulin Delivery Computing Entity

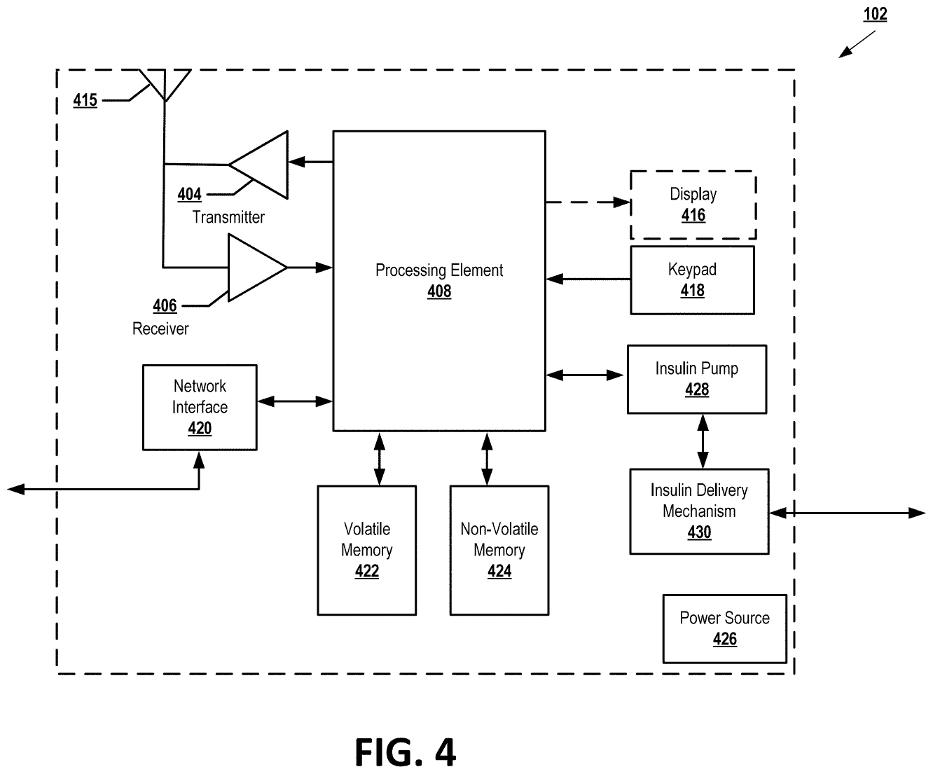

FIG. 4 provides an illustrative schematic representative of an automated insulin delivery computing entity 102 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Automated insulin delivery computing entities 102 can be operated by various parties. As shown in FIG. 4, the automated insulin delivery computing entity 102 can comprise an antenna 412, a transmitter 404 (e.g., radio), a receiver 406 (e.g., radio), a processing element 408 (e.g., CPLDs, microprocessors, multi-core processors, coprocessing entities, ASIPs, microcontrollers, and/or controllers) that provides signals to and receives signals from the transmitter 404 and receiver 406, correspondingly, a power source 426, an insulin pump 428, and an insulin delivery mechanism 430.

The signals provided to and received from the transmitter 404 and the receiver 406, correspondingly, may comprise signaling information/data in accordance with air interface standards of applicable wireless systems. In this regard, the automated insulin delivery computing entity 102 may be capable of operating with one or more air interface standards, communication protocols, modulation types, and access types. More particularly, the automated insulin delivery computing entity 102 may operate in accordance with any of a number of wireless communication standards and protocols, such as those described above with regard to the predictive data analysis computing entity 106. In a particular embodiment, the automated insulin delivery computing entity 102 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the automated insulin delivery computing entity 102 may operate in accordance with multiple wired communication standards and protocols, such as those described above with regard to the predictive data analysis computing entity 106 via a network interface 420.

Via these communication standards and protocols, the automated insulin delivery computing entity 102 can communicate with various other entities using concepts such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The automated insulin delivery computing entity 102 can also download changes, add-ons, and updates, for instance, to its firmware, software (e.g., including executable instructions, applications, program modules), and operating system.

According to one embodiment, the automated insulin delivery computing entity 102 may comprise location determining aspects, devices, modules, functionalities, and/or similar words used herein interchangeably. For example, the automated insulin delivery computing entity 102 may comprise outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This information/data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the automated insulin delivery computing entity's 102 position in connection with a variety of other systems, including cellular towers, Wi-Fi access points, and/or the like. Similarly, the automated insulin delivery computing entity 102 may comprise indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may comprise the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

In some embodiments, the transmitter 404 may include one or more Bluetooth transmitters. In some embodiments, the receiver 406 may include one or more Bluetooth receivers. The Bluetooth transmitters and/or the Bluetooth receivers may be configured to communicate with at least one of the client computing entity 103 and the predictive data analysis computing entity 106. In some embodiments, the transmitter 404 may include one or more WAN transmitters. In some embodiments, the receiver 406 may include one or more WAN receivers. The WAN transmitters and/or the WAN receivers may be configured to communicate with at least one of the client computing entity 103 and the predictive data analysis computing entity 106.

The power source 426 may include electric circuitry configured to enable powering the automated insulin delivery computing entity 102. The power source 426 may include one or more batteries, such as a rechargeable lithium-ion (Li-Ion) battery, that are configured to act as sources of electric power for the automated insulin delivery computing entity 102.

The automated insulin delivery computing entity 102 may also optionally comprise a user interface (that can comprise a display 416 coupled to a processing element 408) and/or a user input interface (coupled to a processing element 408). For example, the user interface may be a user application, browser, user interface, and/or similar words used herein interchangeably executing on and/or accessible via the automated insulin delivery computing entity 102 to interact with and/or cause display of information/data from the predictive data analysis computing entity 106, as described herein. The user input interface can comprise any of a number of devices or interfaces allowing the automated insulin delivery computing entity 102 to receive data, such as a keypad 418 (hard or soft), a touch display, voice/speech or motion interfaces, or other input device. In embodiments including a keypad 418, the keypad 418 can comprise (or cause display of) the conventional numeric (0-9) and related keys (#, *), and other keys used for operating the automated insulin delivery computing entity 102 and may comprise a full plurality of alphabetic keys or plurality of keys that may be activated to provide a full plurality of alphanumeric keys. In addition to providing input, the user input interface can be used, for example, to activate or deactivate certain functions, such as screen savers and/or sleep modes.

The automated insulin delivery computing entity 102 can also comprise volatile storage or memory 422 and/or non-volatile storage or memory 424, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the automated insulin delivery computing entity 102. As indicated, this may comprise a user application that is resident on the entity or accessible through a browser or other user interface for communicating with the predictive data analysis computing entity 106 and/or various other computing entities.

In another embodiment, the automated insulin delivery computing entity 102 may comprise one or more components or functionality that are the same or similar to those of the predictive data analysis computing entity 106, as described in greater detail above. As will be recognized, these architectures and descriptions are provided for exemplary purposes only and are not limiting to the various embodiments.

### Exemplary Client Computing Entity

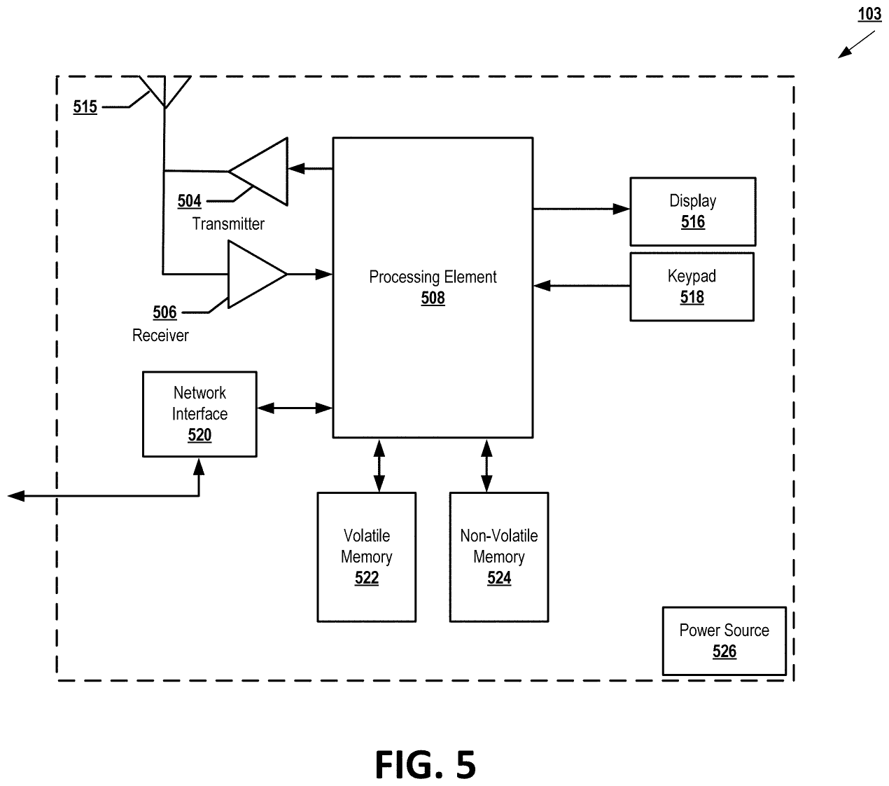

FIG. 5 provides an illustrative schematic representative of a client computing entity 103 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Client computing entities 103 can be operated by various parties. As shown in FIG. 5, the client computing entity 103 can comprise an antenna 512, a transmitter 504 (e.g., radio), a receiver 506 (e.g., radio), a processing element 508 (e.g., CPLDs, microprocessors, multi-core processors, coprocessing entities, ASIPs, microcontrollers, and/or controllers) that provides signals to and receives signals from the transmitter 504 and receiver 506, correspondingly, and a power source 526.

The signals provided to and received from the transmitter 504 and the receiver 506, correspondingly, may comprise signaling information/data in accordance with air interface standards of applicable wireless systems. In this regard, the client computing entity 103 may be capable of operating with one or more air interface standards, communication protocols, modulation types, and access types. More particularly, the client computing entity 103 may operate in accordance with any number of wireless communication standards and protocols, such as those described above with regard to the predictive data analysis computing entity 106. In a particular embodiment, the client computing entity 103 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the client computing entity 103 may operate in accordance with multiple wired communication standards and protocols, such as those described above with regard to the predictive data analysis computing entity 106 via a network interface 520.

Via these communication standards and protocols, the client computing entity 103 can communicate with various other entities using concepts such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The client computing entity 103 can also download changes, add-ons, and updates, for instance, to its firmware, software (e.g., including executable instructions, applications, program modules), and operating system.

According to one embodiment, the client computing entity 103 may comprise location determining aspects, devices, modules, functionalities, and/or similar words used herein interchangeably. For example, the client computing entity 103 may comprise outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This information/data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the glucose monitoring computing entity's 102 position in connection with a variety of other systems, including cellular towers, Wi-Fi access points, and/or the like. Similarly, the client computing entity 103 may comprise indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may comprise the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

In some embodiments, the transmitter 504 may include one or more Bluetooth transmitters. In some embodiments, the receiver 506 may include one or more Bluetooth receivers. The Bluetooth transmitters and/or the Bluetooth receivers may be configured to communicate with at least one of the glucose monitoring computing entity 101 and the automated insulin delivery computing entity 102. In some embodiments, the transmitter 504 may include one or more WAN transmitters. In some embodiments, the receiver 506 may include one or more WAN receivers. The WAN transmitters and/or the WAN receivers may be configured to communicate with the predictive data analysis computing entity 106.

The power source 526 may include electric circuitry configured to enable powering the client computing entity 103. The power source 526 may include one or more batteries, such as a nickel metal-hydride (NiMH) battery, that are configured to act as sources of electric power for the client computing entity 103.

The client computing entity 103 may also comprise a user interface (that can comprise a display 516 coupled to a processing element 508) and/or a user input interface (coupled to a processing element 508). For example, the user interface may be a user application, browser, user interface, and/or similar words used herein interchangeably executing on and/or accessible via the client computing entity 103 to interact with and/or cause display of information/data from the predictive data analysis computing entity 106, as described herein. The user input interface can comprise any of a number of devices or interfaces allowing the client computing entity 103 to receive data, such as a keypad 518 (hard or soft), a touch display, voice/speech or motion interfaces, or other input device. In embodiments including a keypad 518, the keypad 518 can comprise (or cause display of) the conventional numeric (0-9) and related keys (#, *), and other keys used for operating the client computing entity 103 and may comprise a full plurality of alphabetic keys or plurality of keys that may be activated to provide a full plurality of alphanumeric keys. In addition to providing input, the user input interface can be used, for example, to activate or deactivate certain functions, such as screen savers and/or sleep modes.

The client computing entity 103 can also comprise volatile storage or memory 522 and/or non-volatile storage or memory 524, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the client computing entity 103. As indicated, this may comprise a user application that is resident on the entity or accessible through a browser or other user interface for communicating with the predictive data analysis computing entity 106 and/or various other computing entities.

In another embodiment, the client computing entity 103 may comprise one or more components or functionalities that are the same or similar to those of the predictive data analysis computing entity 106, as described in greater detail above. As will be recognized, these architectures and descriptions are provided for exemplary purposes only and are not limiting to the various embodiments.

### Exemplary External Computing Entity

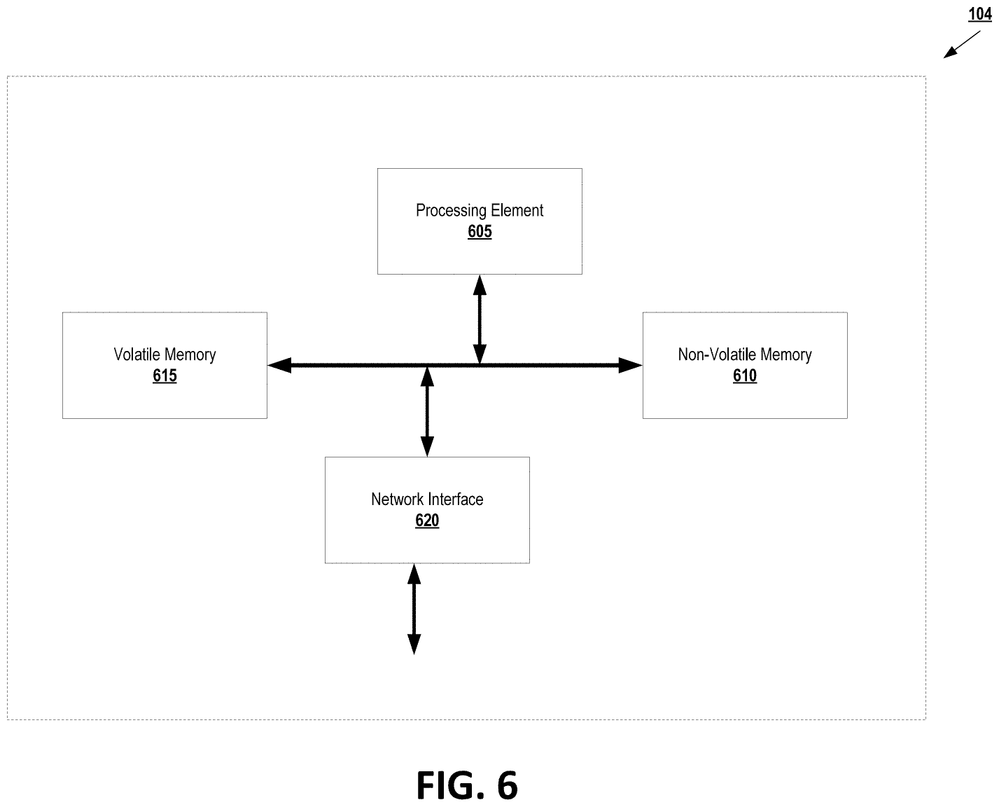

FIG. 6 provides a schematic of an external computing entity 104 according to one embodiment of the present invention. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the predictive data analysis computing entity 106 may also comprise one or more network interfaces 620 for communicating with various computing entities, such as by communicating data, content, information, and/or similar terms used herein interchangeably that can be transmitted, received, operated on, processed, displayed, stored, and/or the like.

As shown in FIG. 6, in one embodiment, the external computing entity 104 may comprise or be in communication with one or more processing elements 605 (also referred to as processors, processing circuitry, and/or similar terms used herein interchangeably) that communicate with other elements within the external computing entity 104 via a bus, for example. As will be understood, the processing element 605 may be embodied in a number of different ways.

For example, the processing element 605 may be embodied as one or more complex programmable logic devices (CPLDs), microprocessors, multi-core processors, coprocessing entities, application-specific instruction-set processors (ASIPs), microcontrollers, and/or controllers. Further, the processing element 605 may be embodied as one or more other processing devices or circuitry. The term circuitry may refer to an entirely hardware embodiment or a combination of hardware and computer program products. Thus, the processing element 605 may be embodied as integrated circuits, application specific integrated circuits (ASICs), field programmable gate arrays (FPGAs), programmable logic arrays (PLAs), hardware accelerators, another circuitry, and/or the like.

As will therefore be understood, the processing element 605 may be configured for a particular use or configured to execute instructions stored in volatile or non-volatile media or otherwise accessible to the processing element 205. As such, whether configured by hardware or computer program products, or by a combination thereof, the processing element 605 may be capable of performing steps or operations according to embodiments of the present invention when configured accordingly.

In one embodiment, the external computing entity 104 may further comprise or be in communication with non-volatile media (also referred to as non-volatile storage, memory, memory storage, memory circuitry and/or similar terms used herein interchangeably). In one embodiment, the non-volatile storage or memory may comprise one or more non-volatile storage or memory media 610, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or information/data that is stored in a computer-readable storage medium using one or more database models, such as a hierarchical database model, network model, relational model, entity-relationship model, object model, document model, semantic model, graph model, and/or the like.

In one embodiment, the external computing entity 104 may further comprise or be in communication with volatile media (also referred to as volatile storage, memory, memory storage, memory circuitry and/or similar terms used herein interchangeably). In one embodiment, the volatile storage or memory may also comprise one or more volatile storage or memory media 615, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the predictive data analysis computing entity 106 with the assistance of the processing element 605 and operating system.

As indicated, in one embodiment, the external computing entity 104 may also comprise one or more network interfaces 620 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive data analysis computing entity 106 may be configured to communicate via wireless client communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the predictive data analysis computing entity 106 may comprise or be in communication with one or more input elements, such as a keyboard input, a mouse input, a touch screen/display input, motion input, movement input, audio input, pointing device input, joystick input, keypad input, and/or the like. The predictive data analysis computing entity 106 may also comprise or be in communication with one or more output elements (not shown), such as audio output, video output, screen/display output, motion output, movement output, and/or the like.

### V. EXEMPLARY METHOD OPERATIONS

Using the techniques described below, various embodiments of the present invention address technical challenges associated with efficiency and effectiveness of performing metabolic predictive data analysis, and enable performing metabolic predictive data analysis on time windows having diverse user activity profiles by utilizing a unified machine learning framework that is configured to adapt to variations in the input structures of diverse prediction windows. Accordingly, by reducing the number of machine learning models that should be utilized to perform effective metabolic predictive data analysis in relation to prediction windows having diverse user activity profiles, various embodiments of the present invention both: (i) improve the computational complexity of performing metabolic predictive data analysis by reducing the need for parallel implementation of multiple machine learning models as well as normalizing the outputs of multiple machine learning models, and (ii) reduce the storage costs of performing metabolic predictive data analysis by eliminating the need to store model definition data (e.g., model parameter data and/or model hyper-parameter data) for multiple machine learning models. Accordingly, by addressing the technical challenges associated with efficiency and effectiveness of performing metabolic predictive data analysis, various embodiments of the present invention make substantial technical contributions to improving efficiency and effectiveness of performing metabolic predictive data analysis and to the field of predictive data analysis generally.

While various embodiments of the present invention describe performing particular operations on data associated with a single monitored individual, a person of ordinary skill in the relevant technology will recognize that all of the operations that are described herein as being performed on data associated with a single monitored individual can be performed on data associated with two or more monitored individuals. In some embodiments, biometric data is used to segment activities and current biometric data is used to allocate probabilities to one or more monitored individuals.

A. Generating Activity Recommendation Machine Learning Models

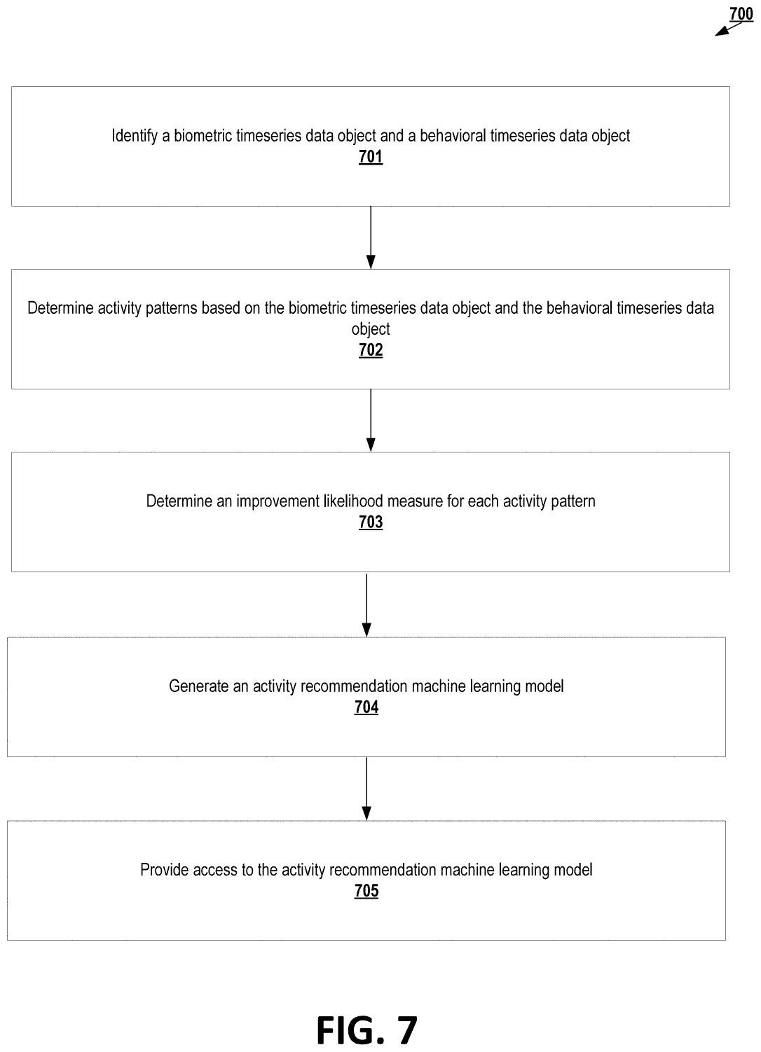

FIG. 7 is a flowchart diagram of an example process 700 for generating an activity recommendation machine learning model. Via the various steps/operations of the process 700, the predictive data analysis computing entity 106 can use historically inferred activity patterns across at least one of historical behavioral timeseries data objects and historical biometric timeseries data objects to generate improvement likelihood measures that are determined based historically observed biometric impacts of the inferred activity patterns.

The process 700 begins at step/operation 701 when the predictive data analysis computing entity 106 identifies a biometric timeseries data object and a behavioral timeseries data object. In some embodiments, the biometric timeseries data object is a historic biometric timeseries data object that can be used to infer one or more activity patterns as well as a biometric impact subset for each activity pattern. In some embodiments, the behavioral timeseries data object is a historic behavioral biometric timeseries data object that aligns temporally with the biometric timeseries data object that is a historic biometric timeseries data object.

In some embodiments, two timeseries data object are deemed to temporally align if at least n (e.g., at least one, or at least a required ratio of) of the corresponding time windows described by the timeseries data objects refer to common periods. For example, in some embodiments, given a historical biometric timeseries data object that includes n biometric time windows and a historical behavioral timeseries data object that includes m behavioral time windows, and given that p of the n biometric time windows correspond to time periods described by the m behavioral time windows, the historical biometric timeseries data object and the historical behavioral timeseries data object may in some embodiments be deemed to temporally align if p satisfies a temporal alignment threshold. As another example, in some embodiments, given a historical biometric timeseries data object that includes n biometric time windows and a historical behavioral timeseries data object that includes m behavioral time windows, and given that p of the m behavioral time windows correspond to time periods described by the n biometric time windows, the historical biometric timeseries data object and the historical behavioral timeseries data object may in some embodiments be deemed to temporally align if p satisfies a temporal alignment threshold. As yet another example, in some embodiments, given a historical biometric timeseries data object that includes n biometric time windows and a historical behavioral timeseries data object that includes m behavioral time windows, and given that p of the n biometric time windows correspond to time periods described by the m behavioral time windows, and further given that q of the m behavioral time windows correspond to time periods described by the n biometric time windows the historical biometric timeseries data object and the historical behavioral timeseries data object may in some embodiments be deemed to temporally align if p satisfies a first temporal alignment threshold and q satisfies a second temporal alignment threshold.

In some embodiments, the behavioral timeseries data object describes a recorded behavioral activity description measure for a monitored individual over a plurality of time periods. For example, in some embodiments, the behavioral timeseries data object may describe a recorded movement velocity of a monitored individual over a plurality of time windows. As another example, in some embodiments, the behavioral timeseries data object may describe a recorded calorie consumption rate of a monitored individual over a plurality of time windows. As yet another example, in some embodiments, the behavioral timeseries data object may describe a recorded pulse rate of a monitored individual over a plurality of time windows. As a further example, in some embodiments, the behavioral timeseries data object may describe a recorded bodily exercise frequency of a monitored individual over a plurality of time windows. In some embodiments, the data described by the behavioral timeseries data object is determined by using one or more behavioral sensor devices that are configured to monitor behavioral conditions of the monitored individual periodically or continuously over time and report the noted behavioral conditions to one or more server computing entities, where the server computing entities are configured to generate the behavioral timeseries data object based at least in part on the behavioral condition data that is received from the noted one or more behavioral sensors. In some embodiments, the behavioral timeseries data object is generated based at least in part on each plurality of recorded observations for an individual of a plurality of individuals, and each plurality of recorded observations for an individual is determined based at least in part on a plurality of observation time windows for the individual, and the plurality of behavioral time windows comprise each plurality of observation time windows for an individual.

In some embodiments, the behavioral timeseries data object is associated with a plurality of behavioral prediction windows, where a prediction window may describe a time period of the plurality of time periods across which a behavioral timeseries data object is calculated. For example, in some embodiments, a plurality of behavioral time windows describes a plurality of defined time periods that follow each other in a continuous manner across which a behavioral timeseries data object is calculated. As another example, in some embodiments, a plurality of behavioral time windows describes a plurality of disjoint time periods across which a behavioral timeseries data object is calculated. As yet another example, in some embodiments, a plurality of behavioral time windows describes: (i) one or more sets of continuous time periods, where each set describes a plurality of defined time periods that follow each other in a continuous manner across which a behavioral timeseries data object is calculated, and (ii) one or more sets of disjoint time periods, where each set describes a plurality of disjoint time periods across which a behavioral timeseries data object is calculated.

In some embodiments, a behavioral timeseries data object is determined based at least in part on a user activity profile for a corresponding monitored individual, where the user activity profile may describe recorded user activity events of a corresponding prediction window and indicates an activity order for the noted recorded user activity events. For example, a particular user activity profile may describe that a corresponding prediction window is associated with the following timeline of events: recorded user activity event A1 is performed prior to recorded user activity event A2, which is in turn performed prior to recorded user activity event A3. As another example, another user activity profile may describe that a corresponding prediction window is associated with the following timeline of events: (i) recorded user activity event A1 is performed closely before recorded user activity event A2, which is in turn performed closely before recorded user activity event A3; and (ii) recorded user activity event A4 is performed long after recorded user activity event A3. As yet another example, another user activity profile may describe that a corresponding prediction window is associated with the following timeline of events: (i) recorded user activity event A1 is performed two hours prior to recorded user activity event A2; (ii) recorded user activity event A2 is performed one hour prior to recorded user activity event A3; (iii) recorded user activity event A3 is performed thirty-four minutes prior to recorded user activity event A4; and (iv) recorded user activity event A4 is performed three hours prior to recorded user activity event A5. An example of a user activity profile is a bolus intake profile that describes a sequential occurrence of one or more recorded user activity event. In some embodiments, the user activity profile includes a plurality of recorded user activity events associated with a prediction window that are separated by sufficient time from one another (e.g., separated by at least a length of time that is equal to the amount of time needed for glucose concentration levels of a monitored individual to return to a baseline glucose concentration level).

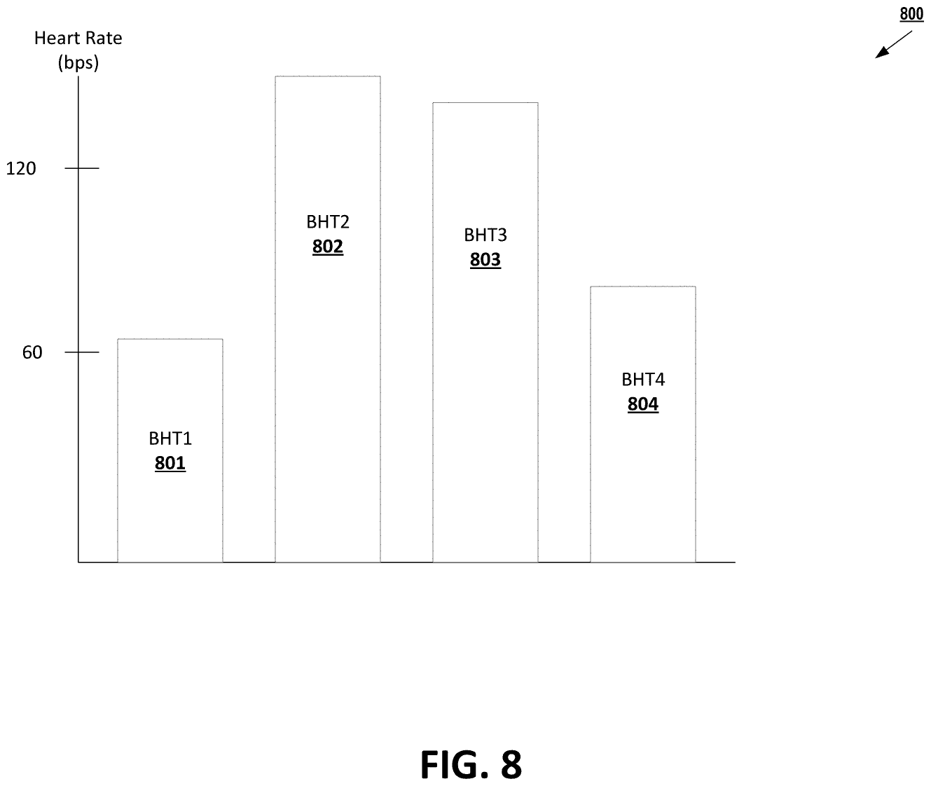

An operational example of a behavioral timeseries data object 800 is depicted in FIG. 8. As depicted in FIG. 8, the behavioral timeseries data object 800 describes a measure of heart rate over a plurality of behavioral time windows BHT1-BHT4 801-804. As depicted in FIG. 8, the heart rate measure peaks across behavioral time windows BHT2-BHT3 802-803, while it returns to a pre-peak level at the behavioral time window BHT4 804.

In some embodiments, a biometric timeseries data object describes a recorded biometric measure for a monitored individual over a plurality of time periods. For example, in some embodiments, the biometric timeseries data object may describe a recorded blood glucose level of a monitored individual over a plurality of time windows. As another example, in some embodiments, the biometric timeseries data object may describe a recorded heart rate of a monitored individual over a plurality of time windows. As yet another example, in some embodiments, the biometric timeseries data object may describe a recorded pulse rate of a monitored individual over a plurality of time windows. As a further example, in some embodiments, the biometric timeseries data object may describe a recorded bodily temperature of a monitored individual over a plurality of time windows. As an additional example, in some embodiments, the biometric timeseries data object may describe a recorded breathing rate of a monitored individual over a plurality of time windows. In some embodiments, the data described by the biometric timeseries data object is determined by using one or more biometric sensor devices that are configured to monitor biometric conditions of the monitored individual periodically or continuously over time and report the noted biometric conditions to one or more server computing entities, where the server computing entities are configured to generate the biometric timeseries data object based at least in part on the biometric condition data that is received from the noted one or more biometric sensors. In some embodiments, the biometric timeseries data object is generated based at least in part on one or more recorded longitudinal observations of a corresponding individual across the plurality of biometric time windows. In some embodiments, the biometric timeseries data object is generated based at least in part on each plurality of recorded observations for an individual of a plurality of individuals, each plurality of recorded observations for an individual is determined based at least in part on a plurality of observation time windows for the individual, and the plurality of biometric time windows comprise each plurality of observation time windows for an individual.

In some embodiments, a biometric timeseries data object is associated with one or more biometric time window, where each biometric time window may describe a time period of the plurality of time periods across which a biometric timeseries data object is calculated. For example, in some embodiments, a plurality of biometric time windows describes a plurality of defined time periods that follow each other in a continuous manner across which a biometric timeseries data object is calculated. As another example, in some embodiments, a plurality of biometric time windows describes a plurality of disjoint time periods across which a biometric timeseries data object is calculated. As yet another example, in some embodiments, a plurality of biometric time windows describes: (i) one or more sets of continuous time periods, where each set describes a plurality of defined time periods that follow each other in a continuous manner across which a biometric timeseries data object is calculated, and (ii) one or more sets of disjoint time periods, where each set describes a plurality of disjoint time periods across which a biometric timeseries data object is calculated.

In some embodiments, a biometric timeseries data object is determined based at least in part on a glucose measurement profile for a monitored individual, where the glucose measurement profile describe one or more recorded glucose concentration measurements (e.g., a portion of the recorded glucose concentration measurements, all of the recorded glucose concentration measurements, and/or the like) for a corresponding prediction window, where each corresponding timestamp for a glucose concentration measurement of the one or more glucose concentration measurements falls within a period of time described by the prediction window. In some embodiments, the timestamp of a glucose concentration measurement is determined based at least in part on a measurement time of the glucose concentration measurement. In some embodiments, a timestamp of a glucose concentration measurement is determined based at least in part on an adjusted measurement time of the glucose concentration measurement, wherein the adjusted measurement time may be determined by adjusting the measurement time of the glucose concentration measurement by a glucose concentration peak interval. In some embodiments, the glucose concentration measurements described by the glucose measurement profile may be determined using continuous glucose monitoring.

In some embodiments, a biometric timeseries data object is determined based at least in part on a glucose measurement timeseries data object, where the glucose measurement timeseries data object may be configured to describe selected recorded glucose concentration measurements associated with a corresponding prediction window, where the selected recorded glucose concentration measurements are deemed related to (e.g., have timestamps that occur within a predefined time interval subsequent to, such as within 3-5 hours subsequent to) at least one recorded user activity event of a user activity profile. For example, a glucose concentration measurement timeseries data object may describe that a corresponding prediction window is associated with the following timeline of selected glucose concentration measurements: recorded glucose measurement M1 is performed prior to recorded glucose measurement M2, which is in turn performed prior to recorded glucose measurement M3. As another example, another glucose concentration measurement timeseries data object may describe that a corresponding prediction window is associated with the following timeline of selected glucose concentration measurements: (i) recorded glucose measurement M1 is performed closely before recorded glucose measurement M2, which is in turn performed closely before recorded glucose measurement M3; and (ii) recorded glucose measurement M4 is performed long after recorded glucose measurement M4. As yet another example, another glucose concentration measurement timeseries data object may describe that a corresponding prediction window is associated with the following timeline of selected glucose concentration measurements: (i) recorded glucose measurement M1 is performed three hours prior to recorded glucose measurement M2; (i) recorded glucose measurement M2 is performed two hours prior to recorded glucose measurement M3; (iii) recorded glucose measurement M3 is performed thirty-eight minutes prior to recorded glucose measurement M4; and (iv) recorded glucose measurement M4 is performed two hours prior to recorded glucose measurement M5. In some embodiments, the measurement timeseries data object describes the recorded glucose measurements along with one or more extrapolated glucose measurements inferred using one or more temporal extrapolation techniques to fill in the gaps between the noted recorded glucose concentration measurements.

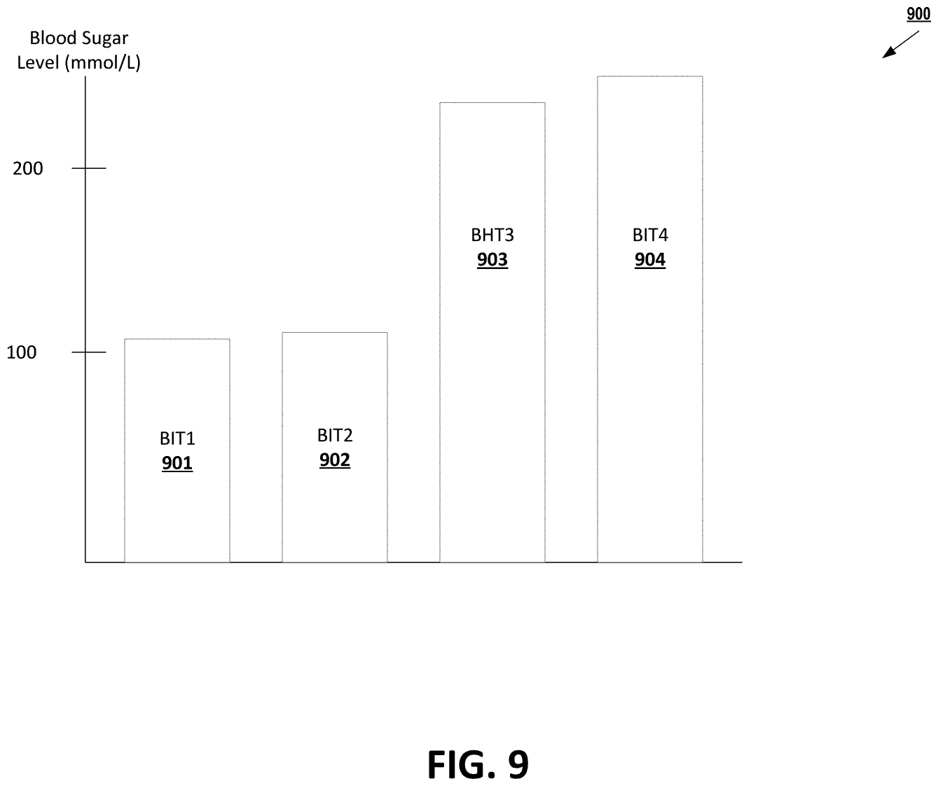

An operational example of a biometric timeseries data object 900 is depicted in FIG. 9. As depicted in FIG. 9, the biometric timeseries data object 900 describes a measure of blood glucose level over a plurality of biometric time windows BIT1-BIT4 901-904, which corresponds to behavioral time windows BHT1-BHT4 801-804 of FIG. 8. As depicted in FIG. 9, the blood glucose level measure peaks across biometric time windows BIT3-BIT4 902-903.

At step/operation 702, the predictive data analysis computing entity 106 determines one or more activity patterns based at least in part on the biometric timeseries data object and the behavioral timeseries data object. In some embodiments, the predictive data analysis computing entity 106 determines each activity pattern based at least in part on an occurrence detection time window set for the activity pattern, as the term is defined below. In some embodiments, the occurrence detection time window set for an activity pattern includes at least one of a behavioral occurrence detection time window subset of the plurality of behavioral time windows or a biometric occurrence detection time window subset of the plurality of biometric time windows.

In some embodiments, an occurrence detection window subset includes a plurality of time windows that are deemed to describe an activity pattern. In some embodiments, the occurrence detection window subset may include a plurality of behavioral time windows that are deemed to describe an activity pattern. For example, the occurrence detection time window subset may include a plurality of behavioral time windows that are deemed to describe an activity pattern as determined based at least in part on behavioral description measures associated with the behavioral time window. In some embodiments, the occurrence detection window subset may include a plurality of biometric time windows that are deemed to describe an activity pattern. For example, the occurrence detection time window subset may include a plurality of biometric time windows that are deemed to describe an activity pattern as determined based at least in part on biometric measures associated with the plurality of biometric time windows. For example, the occurrence detection window subset may include a plurality of behavioral time windows and a plurality of biometric time windows, where correlating the behavioral description measures of the plurality of behavioral time windows and the biometric measures of the plurality of biometric time windows indicates that the plurality of behavioral time windows and the plurality of biometric time windows collectively describe a detected/identified activity. In some embodiments, if an activity pattern is detected solely based at least in part on behavioral data (e.g., based at least in part on behavioral timeseries data objects), then the occurrence detection window set for that activity pattern includes any time periods in the behavioral data that are used to detect an activity pattern. In some embodiments, if an activity pattern is detected solely based at least in part on biometric data (e.g., based at least in part on biometric timeseries data objects), then the occurrence detection window set for that activity pattern includes any time periods in the biometric data that are used to detect an activity pattern. In some embodiments, if an activity pattern is detected based at least in part on both behavioral data and biometric data (e.g., based at least in part on behavioral timeseries data objects and biometric timeseries data objects), then the occurrence detection window set for that activity pattern includes any time periods in the behavioral data and any time periods in the biometric data that are used to detect the activity pattern, where the time periods in the behavioral data and the time periods in the biometric data are deemed to be temporally correlated in a manner that are deemed to refer to the same activity pattern.

As described above, in some embodiments, the occurrence detection time window set for an activity pattern includes a behavioral occurrence detection time window subset of the plurality of behavioral time windows. In some embodiments, a behavioral occurrence detection time window subset may include a plurality of behavioral time windows that are deemed to describe an activity pattern. In some embodiments, the occurrence detection window subset may include a plurality of behavioral time windows that are deemed to describe an activity pattern. For example, the occurrence detection time window subset may include a plurality of behavioral time windows that are deemed to describe an activity pattern as determined based at least in part on behavioral description measures associated with the behavioral time window. In some embodiments, if an activity pattern is detected solely based at least in part on behavioral data (e.g., based at least in part on behavioral timeseries data objects), then the occurrence detection window set for that activity pattern includes any time periods in the behavioral data that are used to detect an activity pattern. Examples of behavioral occurrence detection time window subsets include sets of behavioral time windows that describe intensive physical activity patterns, intense exercise activity patterns, substantial food intake activity patterns, fasting activity patterns, and/or the like. For example, in some embodiments, a plurality of behavioral time windows may be in a behavioral occurrence detection time window subset if monitored behavioral activity measures (e.g., movement velocity measures, heart rate measures, and/or the like) for the plurality of behavioral time windows (e.g., as described by a behavioral timeseries data object) describe that a monitored individual has engaged in a desired/target activity pattern (e.g., running, exercise, and/or the like).

As described above, in some embodiments, the occurrence detection time window set for an activity pattern includes a behavioral occurrence detection time window subset of the plurality of behavioral time windows or a biometric occurrence detection time window subset of the plurality of biometric time windows. In some embodiments, a behavioral occurrence detection time window subset may include a plurality of biometric time windows that are deemed to describe an activity pattern. In some embodiments, the occurrence detection window subset may include a plurality of biometric time windows that are deemed to describe an activity pattern. For example, the occurrence detection time window subset may include a plurality of biometric time windows that are deemed to describe an activity pattern as determined based at least in part on biometric measures associated with the plurality of biometric time windows. In some embodiments, if an activity pattern is detected solely based at least in part on behavioral data (e.g., based at least in part on behavioral timeseries data objects), then the occurrence detection window set for that activity pattern includes any time periods in the behavioral data that are used to detect an activity pattern. In some embodiments, if an activity pattern is detected solely based at least in part on biometric data (e.g., based at least in part on biometric timeseries data objects), then the occurrence detection window set for that activity pattern includes any time periods in the biometric data that are used to detect an activity pattern. Examples of biometric occurrence detection time window subsets include sets of biometric time windows that describe intensive physical activity patterns, intense exercise activity patterns, substantial food intake activity patterns, fasting activity patterns, and/or the like. For example, in some embodiments, a plurality of biometric time windows may be in a biometric occurrence detection time window subset if monitored biometric measures (e.g., glucose levels, heart rates, breathing rates, etc.) associated with the plurality of biometric time windows (e.g., as described by a biometric timeseries data object) describe that a monitored individual has engaged in a desired/target activity pattern (e.g., calorie intake, running, exercise, and/or the like). As another example, in some embodiments, a plurality of biometric time windows may be in a biometric occurrence detection time window subset if glucose levels associated with the plurality of biometric time windows describe that a monitored individual has performed a calorie intake. As yet another example, in some embodiments, a plurality of biometric time windows may be in a biometric occurrence detection time window subset if breathing rates and/or heart rates associated with the plurality of biometric time windows describe that a monitored individual has engaged in intense physical activity. As an additional example, in some embodiments, a plurality of biometric time windows may be in a biometric occurrence detection time window subset if breathing rates and/or heart rates associated with the plurality of biometric time windows describe that a monitored individual has engaged in high-stress activity.

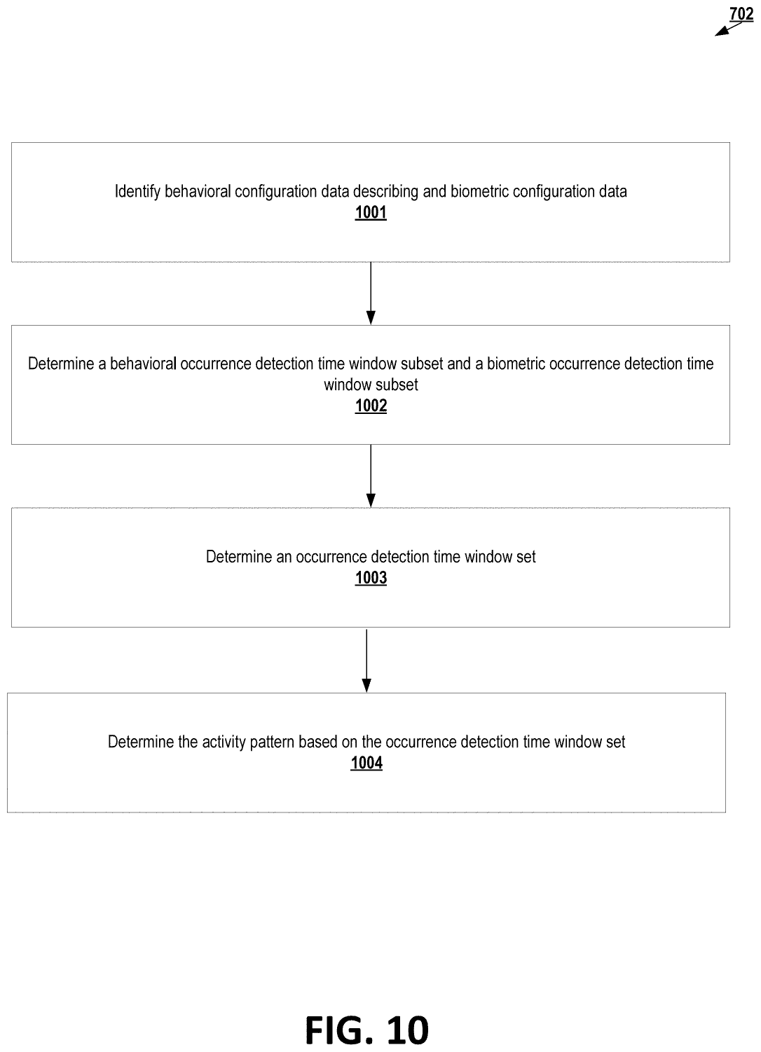

In some embodiments, step/operation 702 may be performed in accordance with the process that is depicted in FIG. 10, which is an example process for determining an activity pattern based at least in part on correlations across a behavioral timeseries data object and a biometric timeseries data object. The process that is depicted in FIG. 10 begins at step/operation 1001 when the predictive data analysis computing entity 106 identifies behavioral configuration data describing how the activity pattern manifests itself in behavioral timeseries data objects as well as biometric configuration data describing how the activity pattern manifests itself in biometric timeseries data objects. For example, the behavioral configuration data may describe that the activity pattern manifests itself as a peak of at least n and at most m behavioral time windows in behavioral timeseries data objects. As another example, the biometric configuration data may describe that the activity pattern manifests itself as a peak of at least p and at most q behavioral time windows in biometric timeseries data objects.

At step/operation 1002, the predictive data analysis computing entity 106 determines a behavioral occurrence detection time window subset of the plurality of behavioral time windows and a biometric occurrence detection time window subset of the plurality of biometric time windows. Examples of behavioral occurrence detection time window subsets include sets of behavioral time windows that describe intensive physical activity patterns, intense exercise activity patterns, substantial food intake activity patterns, fasting activity patterns, and/or the like. For example, in some embodiments, a plurality of behavioral time windows may be in a behavioral occurrence detection time window subset if monitored behavioral activity measures (e.g., movement velocity measures, heart rate measures, and/or the like) for the plurality of behavioral time windows (e.g., as described by a behavioral timeseries data object) describe that a monitored individual has engaged in a desired/target activity pattern (e.g., running, exercise, and/or the like).

In some embodiments, to determine the behavioral occurrence detection time window subset for an activity pattern, the predictive data analysis computing entity 106 identifies a plurality of behavioral time windows of a behavioral timeseries data object that corresponds to the pattern described by the behavioral configuration data. For example, given the behavioral timeseries data object 800 of FIG. 8, and given that behavioral configuration data describes a pattern of two consecutive peak behavioral time windows, the combination of the behavioral time windows BHT2-BHT3 802-803 may constitute the behavioral occurrence detection time window subset.

In some embodiments, to determine the biometric occurrence detection time window subset for an activity pattern, the predictive data analysis computing entity 106 identifies a plurality of biometric time windows of a biometric timeseries data object that corresponds to the pattern described by the biometric configuration data. For example, given the biometric timeseries data object 900 of FIG. 9, and given that biometric configuration data describes a pattern of two consecutive peak behavioral time windows, the combination of the biometric time windows BIT3-BIT4 803-804 may constitute the biometric occurrence detection time window subset.

At step/operation 1003, the predictive data analysis computing entity 106 determines an occurrence detection time window set based at least in part on the behavioral occurrence detection time window subset and the biometric occurrence detection time window subset. In some embodiments, the activity patterns include one or more of the following: (i) biometric activity patterns that are determined solely based at least in part on detected patterns in biometric timeseries data objects (e.g., such that the occurrence detection time window set for each biometric activity pattern comprises the biometric occurrence detection time window subset for the biometric activity pattern), (ii) behavioral activity patterns that are determined solely based at least in part on detected patterns in behavioral timeseries data objects (e.g., such that the occurrence detection time window set for each behavioral activity pattern comprises the behavioral occurrence detection time window subset for the behavioral activity pattern), and (iii) behavioral-biometric activity patterns that are determined based at least in part on detected patterns in correlation data inferred by correlating one or more behavioral timeseries data objects and one or more biometric timeseries data objects (e.g., such that the occurrence detection time window set for each behavioral-biometric activity pattern comprises both the behavioral occurrence detection time window subset for the behavioral-biometric activity pattern and the biometric occurrence detection time window subset for the behavioral-biometric activity pattern, and each behavioral-biometric activity pattern is determined based at least in part on one or more detected cross-timeseries correlations across the plurality of behavioral time windows and the plurality of biometric time windows).

In some embodiments, to determine an occurrence detection time window set based at least in part on the behavioral occurrence detection time window subset and the biometric occurrence detection time window subset, the predictive data analysis computing entity 106 first determines whether the behavioral occurrence detection time window subset and the biometric occurrence detection time window subset are temporally correlated such that they can both be deemed to relate to a common activity pattern. In some embodiments, to determine the noted temporal correlation, the predictive data analysis computing entity 106 uses correlation configuration data that describe what degree/type of temporal correlation between the behavioral occurrence detection time window subset and the biometric occurrence detection time window subset is associated with the activity pattern.

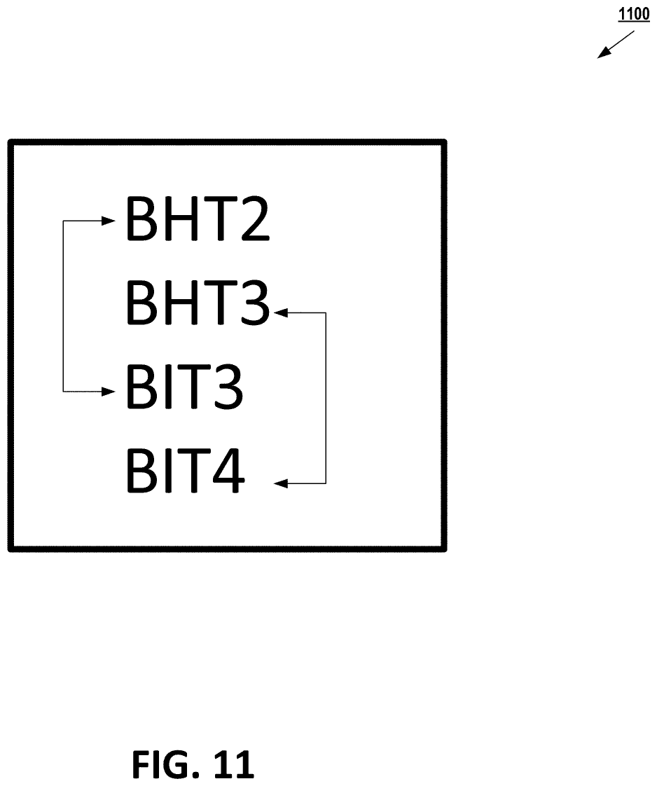

For example, given a behavioral occurrence detection time window subset that includes behavioral time windows BHT2-BHT3 802-803 of FIG. 8, and given a biometric occurrence detection time window subset that includes biometric time windows BIT3-BIT4 903-904 of FIG. 9, and further given correlation configuration data that describes that the biometric occurrence detection time window subset for the corresponding activity pattern should begin within one time window of the termination of the behavioral occurrence detection time window subset of the corresponding activity pattern, the predictive data analysis computing entity 106 may determine that the corresponding activity pattern is associated with an occurrence detection time window set that comprises behavioral time windows BHT2-BHT3 802-803 and biometric time windows BIT3-BIT4 903-904. An operational example of such an occurrence detection time window set 1100 is depicted in FIG. 11.

At step/operation 1004, the predictive data analysis computing entity 106 determines the activity pattern based at least in part on the occurrence detection time window set. In some embodiments, an activity pattern describes a designation that may be associated with an occurrence detection time window set based at least in part on at least one of the following: (i) detected patterns in behavioral timeseries data objects, (ii) detected patterns in biometric timeseries data objects, and (iii) detected patterns in correlation data inferred by correlating one or more behavioral timeseries data objects and one or more biometric timeseries data objects. Examples of activity patterns include designations that describe performing intense physical activities, performing calorie intake activities, performing physical exercise activities, and/or the like.

Returning to FIG. 7, at step/operation 703, the predictive data analysis computing entity 106 determines an improvement likelihood measure for each activity pattern. In some embodiments, the improvement likelihood measure for an activity pattern is determined based at least in part on each desired outcome indicator for a biometric time window that is in the biometric impact subset for the activity pattern, as further described below.

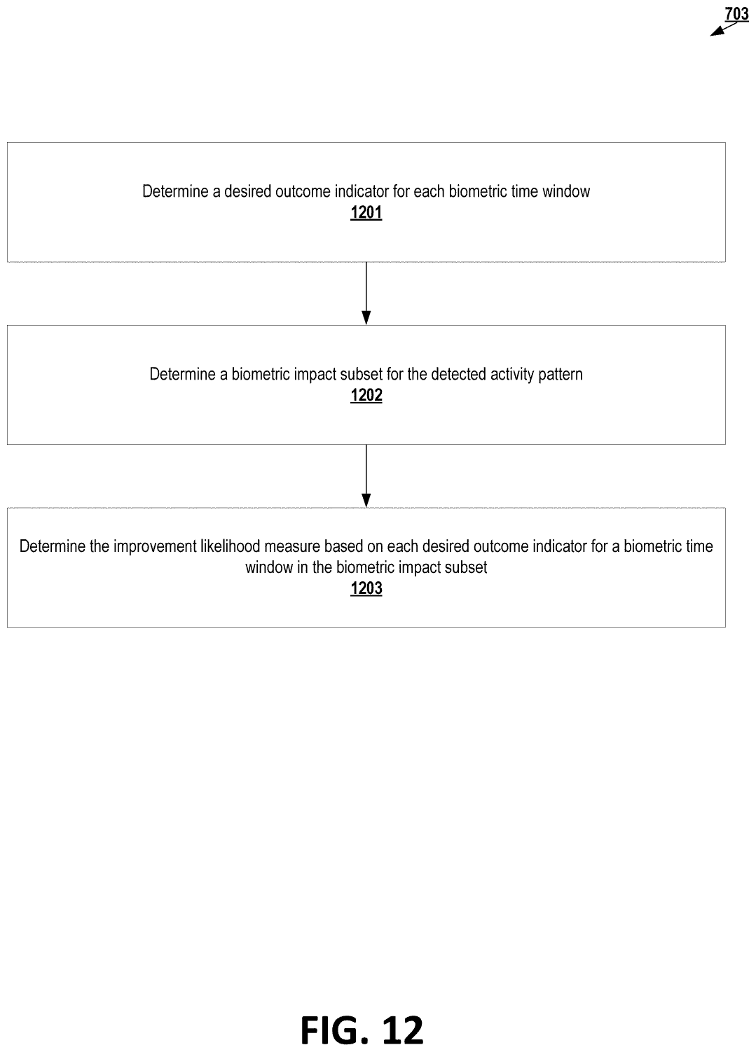

In some embodiments, step/operation 703 may be performed in accordance with the process that is depicted in FIG. 12, which is an example process for generating an improvement likelihood measure for an activity pattern. The process that is depicted in FIG. 12 begins at step/operation 1201 when the predictive data analysis computing entity 106 determines a desired outcome indicator for each biometric time window in the biometric timeseries data object. In some embodiments, the desired outcome indicator for a time window describes if a time window is associated with a biometric condition that is deemed to be a target biometric condition that a predictive data analysis framework is configured to detect.

For example, the desired outcome indicator for a time window may be determined based at least in part on whether a biometric measure for the time window has a value that falls within a threshold range for the biometric measure. As another example, the desired outcome indicator for a time window may be determined based at least in part on whether the time-in-range of the blood glucose level for the time window satisfies a threshold time-in-range condition, where the time-in-range of the blood glucose level for a time window may describe a ratio of the time that the blood glucose level for the time window is within a target range (e.g., a target range deemed to indicate abnormal and/or critical blood glucose level). In some embodiments, a predictive data analysis computing entity determines a desired outcome indicator for each biometric time window based at least in part on whether the biometric measure described for the biometric time window by a biometric timeseries data object falls within a threshold range for the biometric measure. For example, the predictive data analysis computing entity may determine a desired outcome indicator for each biometric time window based at least in part on whether the blood glucose level for the biometric time window by a biometric timeseries data object falls within a threshold range for the blood glucose level. As another example, the predictive data analysis computing entity may determine a desired outcome indicator for each biometric time window based at least in part on whether the recorded heartrate for the biometric time window by a biometric timeseries data object falls within a threshold range for the recorded heartrate. As yet another example, the predictive data analysis computing entity may determine a desired outcome indicator for each biometric time window based at least in part on whether the recorded breathing rate for the biometric time window by a biometric timeseries data object falls within a threshold range for the recorded breathing rate. In some embodiments, each desired outcome indicator for a biometric time window is a target time in range measure for the corresponding biometric time window.

At step/operation 1202, the predictive data analysis computing entity 106 determines a biometric impact subset for the activity pattern. In some embodiments, the activity pattern includes a plurality of time windows that describe biometric impact data describing biometric impacts of an activity pattern. In some embodiments, while the occurrence detection time window subset includes a plurality of time windows that are deemed to describe occurrence of an activity pattern, the biometric impact subset of the activity pattern includes a plurality of biometric time windows that are deemed to describe biometric impacts of an activity pattern. For example, if the occurrence detection time window subset for an activity pattern includes time windows t₁-t₄, and if the biometric impact subset for the activity pattern is deemed to begin n time windows after the termination of the occurrence detection time window subset and last for m time windows, then the biometric impact subset for the activity pattern may include the time windows t₄₊ₙ tot₄₊ₙ₊ₘ. In some embodiments, in the described example, at least one of n and m may be determined (e.g., based at least in part on historical activity monitoring data) in accordance with an activity pattern type of the corresponding activity pattern. In some embodiments, each activity pattern is associated with a plurality of time windows in the biometric data where a proposed system can see the impact of the activity pattern in terms of the desired outcome variable. In some of the noted embodiments, this plurality of time windows in the glucose data is referred to as the biometric impact subset for the activity pattern.

At step/operation 1203, the predictive data analysis computing entity 106 determines the improvement likelihood measure based at least in part on each desired outcome indicator for a biometric time window that is in the biometric impact subset for the activity pattern. The improvement likelihood measure may describe a measure of the likelihood that occurrence of an activity pattern is likely to cause a biometric condition that is deemed to be a target biometric condition that a predictive data analysis framework is configured to detect. In some embodiments, the improvement likelihood measure for an activity pattern is determined based at least in part on the biometric impact subset for the activity pattern, e.g., based at least in part on whether the desired outcome indicators for at least n (e.g., at least one) biometric time windows in the biometric impact subset for the activity pattern describe that the biometric time window is associated with a biometric condition that is deemed to be a target biometric condition that a predictive data analysis framework is configured to detect, or based at least in part on how many desired outcome indicators for biometric time windows in the biometric impact subset for the activity pattern describe that the biometric time window is associated with a biometric condition that is deemed to be a target biometric condition that a predictive data analysis framework is configured to detect. For example, an activity pattern may be associated with an improvement likelihood measure that describes how many of the biometric time windows in the biometric impact subset for the activity pattern are associated with a corresponding desired outcome indicator that describes that the biometric time window is likely to cause a biometric condition that is deemed to be a target biometric condition that a particular predictive data analysis framework is configured to detect.

In some embodiments, if an activity pattern is associated with a biometric impact subset including n biometric time windows, where m of the n biometric time windows are deemed likely to cause a biometric condition that is deemed to be a target biometric condition that a predictive data analysis framework is configured to detect, and n−m of the biometric time windows are deemed unlikely to cause a biometric condition that is deemed to be a target biometric condition that a predictive data analysis framework is configured to detect, then the improvement likelihood measure for the activity pattern is m. In some embodiments, if an activity pattern is associated with a biometric impact subset including n biometric time windows, where m of the n biometric time windows are deemed likely to cause a biometric condition that is deemed to be a target biometric condition that a predictive data analysis framework is configured to detect, and n−m of the biometric time windows are deemed unlikely to cause a biometric condition that is deemed to be a target biometric condition that a predictive data analysis framework is configured to detect, then the improvement likelihood measure for the activity pattern is m n. In some embodiments, if an activity pattern is associated with a biometric impact subset including n biometric time windows, where m of the n biometric time windows are deemed likely to cause a biometric condition that is deemed to be a target biometric condition that a predictive data analysis framework is configured to detect, and n−m of the biometric time windows are deemed unlikely to cause a biometric condition that is deemed to be a target biometric condition that a predictive data analysis framework is configured to detect, then the improvement likelihood measure for the activity pattern is (n−m) n.

Returning to FIG. 7, at step/operation 704, the predictive data analysis computing entity 106 generates an activity recommendation machine learning model. In some embodiments, the activity recommendation machine learning maps each activity pattern to the occurrence detection time window set for the activity pattern and the improvement likelihood measure for the activity pattern.

In some embodiments, the activity recommendation machine learning model associates each activity pattern of a plurality of activity patterns to at least one of the following: (i) an occurrence detection time window set for the activity pattern, and (ii) an improvement likelihood measure for the activity pattern. In some embodiments, the activity recommendation machine learning model maps each activity pattern to the occurrence detection time window set for the activity pattern and the improvement likelihood measure for the activity pattern. In some embodiments, by using an activity recommendation machine learning model, a predictive data analysis computing entity can: (i) process an input behavioral timeseries data object for a monitored individual and/or an input biometric timeseries data object for a monitored individual in order to determine one or more activity patterns in the noted input data objects based at least in part on at least one of the input behavioral timeseries data object, the input biometric timeseries data object, and correlating the input biometric timeseries data object and the input behavioral timeseries data object, (ii) determine the improvement likelihood measures for the activity patterns in the noted input data objects to select a selected subset of the noted activity patterns (e.g., to select the top n activity patterns having the top n improvement likelihood measures, to select the activity patterns whose improvement likelihood measures satisfy an improvement likelihood measure, and/or the like), and (iii) present the selected subset of the noted activity patterns to an end user of the predictive data analysis computing entity. In some embodiments, mappings between activity patterns and occurrence detection time window sets as described by the activity recommendation machine learning model can be used to infer activity patterns based at least in part on input behavioral timeseries data objects and input biometric timeseries data objects. In some embodiments, mappings between activity patterns and improvement likelihood measures can be used to select a selected subset of inferred detectivity patterns, where the inferred activity patterns may be inferred based at least in part on input behavioral timeseries data objects and input biometric timeseries data objects in accordance with mappings between activity patterns and occurrence detection time window sets. In some embodiments, a predictive data analysis computing entity is configured to provide access to the activity recommendation machine learning model, wherein the activity recommendation machine learning model is configured to determine, based at least in part on an input behavioral timeseries data object and an input biometric timeseries data object, a recommended activity pattern subset of the plurality of activity patterns.

At step/operation 705, the predictive data analysis computing entity 106 provides access to the activity recommendation machine learning model. In some embodiments, the activity recommendation machine learning model is configured to determine, based at least in part on an input behavioral timeseries data object and an input biometric timeseries data object, a recommended activity pattern subset of the one or more activity patterns. In some embodiments, the activity recommendation machine learning model is configured to perform a plurality of defined prediction-based actions. In some embodiments, performing the one or more prediction-based actions comprises generating a glucose-insulin prediction for a monitored individual and performing an action based at least in part on the glucose-insulin prediction. A glucose-insulin prediction may describe a conclusion about one or more functional properties of the glucose-insulin endocrine metabolic regulatory system of a corresponding monitored individual. For example, the predictive data analysis computing entity 106 may determine an insulin sensitivity prediction based at least in part on at least one of the maximal insulin secretion rate value and the insulin secretion acceleration value. In some embodiments, if the maximal insulin secretion rate parameter is higher than an expected amount, a computer system may determine that the insulin-dependent glucose-utilizing cells of the monitored individual have developed abnormal levels of insulin sensitivity, which in turn may be used to facilitate an automated diagnosis of type-2 diabetes. As another example, the predictive data analysis computing entity 106 may detect a potential liver problem based at least in part on an abnormally hepatic glucose production parameter. As yet another example, the predictive data analysis computing entity 106 may detect a potential nervous system problem if the insulin-independent glucose uptake rate parameter is abnormally low.

As described above, various embodiments of the present invention address technical challenges associated with efficiency and effectiveness of performing metabolic predictive data analysis, and enable performing metabolic predictive data analysis on time windows having diverse user activity profiles by utilizing a unified machine learning framework that is configured to adapt to variations in the input structures of diverse prediction windows. Accordingly, by reducing the number of machine learning models that should be utilized to perform effective metabolic predictive data analysis in relation to prediction windows having diverse user activity profiles, various embodiments of the present invention both: (i) improve the computational complexity of performing metabolic predictive data analysis by reducing the need for parallel implementation of multiple machine learning models as well as normalizing the outputs of multiple machine learning models, and (ii) reduce the storage costs of performing metabolic predictive data analysis by eliminating the need to store model definition data (e.g., model parameter data and/or model hyper-parameter data) for multiple machine learning models. Accordingly, by addressing the technical challenges associated with efficiency and effectiveness of performing metabolic predictive data analysis, various embodiments of the present invention make substantial technical contributions to improving efficiency and effectiveness of performing metabolic predictive data analysis and to the field of predictive data analysis generally.

B. Predictive Metabolic Intervention Using Activity Recommendation Machine Learning Models

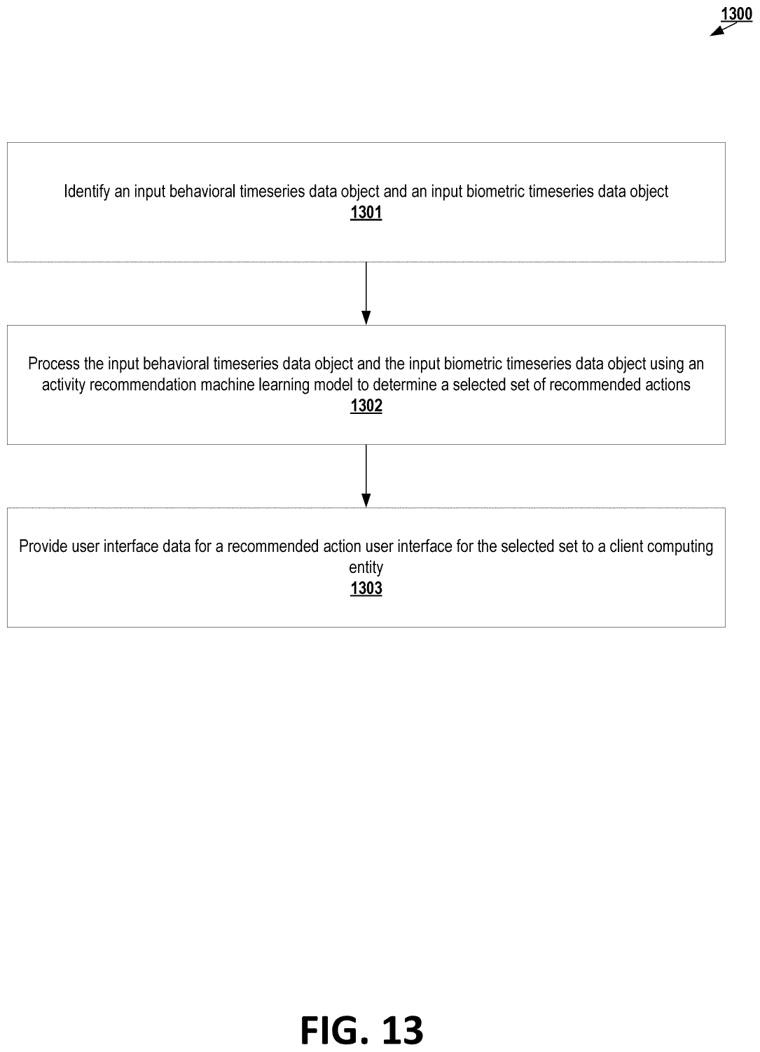

FIG. 13 is a flowchart diagram of an example process 1300 for performing predictive metabolic intervention using an activity recommendation machine learning model, in accordance with some embodiments discussed herein. Via the various steps/operations of the process 1300, the predictive data analysis computing entity 106 can relate activity patterns inferred based at least in part on at least one of behavioral timeseries data objects and biometric timeseries data objects to improvement likelihood measures that are determined based historically observed biometric impacts of the inferred activity patterns.

The process 1300 begins at step/operation 1301 when the predictive data analysis computing entity 106 identifies an input behavioral timeseries data object and an input biometric timeseries data object. The input behavioral timeseries data object and the input biometric timeseries data object can describe respective behavioral data and biometric data of a monitored individual with respect to whom the predictive data analysis computing entity 106 seeks to obtain one or more recommended prediction-based actions.

As described above, the behavioral timeseries data object describes a recorded behavioral activity description measure for a monitored individual over a plurality of time periods. For example, in some embodiments, the behavioral timeseries data object may describe a recorded movement velocity of a monitored individual over a plurality of time windows. As another example, in some embodiments, the behavioral timeseries data object may describe a recorded calorie consumption rate of a monitored individual over a plurality of time windows. As yet another example, in some embodiments, the behavioral timeseries data object may describe a recorded pulse rate of a monitored individual over a plurality of time windows. As a further example, in some embodiments, the behavioral timeseries data object may describe a recorded bodily exercise frequency of a monitored individual over a plurality of time windows. In some embodiments, the data described by the behavioral timeseries data object is determined by using one or more behavioral sensor devices that are configured to monitor behavioral conditions of the monitored individual periodically or continuously over time and report the noted behavioral conditions to one or more server computing entities, where the server computing entities are configured to generate the behavioral timeseries data object based at least in part on the behavioral condition data that is received from the noted one or more behavioral sensors. In some embodiments, the behavioral timeseries data object is generated based at least in part on each plurality of recorded observations for an individual of a plurality of individuals, and each plurality of recorded observations for an individual is determined based at least in part on a plurality of observation time windows for the individual, and the plurality of behavioral time windows comprise each plurality of observation time windows for an individual.

In some embodiments, a behavioral timeseries data object is determined based at least in part on a user activity profile for a corresponding monitored individual, where the user activity profile may describe recorded user activity events of a corresponding prediction window and indicates an activity order for the noted recorded user activity events. For example, a particular user activity profile may describe that a corresponding prediction window is associated with the following timeline of events: recorded user activity event A1 is performed prior to recorded user activity event A2, which is in turn performed prior to recorded user activity event A3. As another example, another user activity profile may describe that a corresponding prediction window is associated with the following timeline of events: (i) recorded user activity event A1 is performed closely before recorded user activity event A2, which is in turn performed closely before recorded user activity event A3; and (ii) recorded user activity event A4 is performed long after recorded user activity event A3. As yet another example, another user activity profile may describe that a corresponding prediction window is associated with the following timeline of events: (i) recorded user activity event A1 is performed two hours prior to recorded user activity event A2; (ii) recorded user activity event A2 is performed one hour prior to recorded user activity event A3; (iii) recorded user activity event A3 is performed thirty-four minutes prior to recorded user activity event A4; and (iv) recorded user activity event A4 is performed three hours prior to recorded user activity event A5. An example of a user activity profile is a bolus intake profile that describes a sequential occurrence of one or more recorded user activity event. In some embodiments, the user activity profile includes a plurality of recorded user activity events associated with a prediction window that are separated by sufficient time from one another (e.g., separated by at least a length of time that is equal to the amount of time needed for glucose concentration levels of a monitored individual to return to a baseline glucose concentration level).

In some embodiments, the input behavioral timeseries data object and the input biometric timeseries data object are temporally aligned. two timeseries data object are deemed to temporally align if at least n (e.g., at least one, or at least a required ratio of) of the corresponding time windows described by the timeseries data objects refer to common periods. For example, in some embodiments, given a historical biometric timeseries data object that includes n biometric time windows and a historical behavioral timeseries data object that includes m behavioral time windows, and given that p of the n biometric time windows correspond to time periods described by the m behavioral time windows, the historical biometric timeseries data object and the historical behavioral timeseries data object may in some embodiments be deemed to temporally align if p satisfies a temporal alignment threshold. As another example, in some embodiments, given a historical biometric timeseries data object that includes n biometric time windows and a historical behavioral timeseries data object that includes m behavioral time windows, and given that p of the m behavioral time windows correspond to time periods described by the n biometric time windows, the historical biometric timeseries data object and the historical behavioral timeseries data object may in some embodiments be deemed to temporally align if p satisfies a temporal alignment threshold. As yet another example, in some embodiments, given a historical biometric timeseries data object that includes n biometric time windows and a historical behavioral timeseries data object that includes m behavioral time windows, and given that p of the n biometric time windows correspond to time periods described by the m behavioral time windows, and further given that q of the m behavioral time windows correspond to time periods described by the n biometric time windows the historical biometric timeseries data object and the historical behavioral timeseries data object may in some embodiments be deemed to temporally align if p satisfies a first temporal alignment threshold and q satisfies a second temporal alignment threshold.

At step/operation 1302, the predictive data analysis computing entity 106 processes the input behavioral timeseries data object and the input biometric timeseries data object using an activity detection machine learning model to determine a selected plurality of recommended actions. In some embodiments, by using an activity recommendation machine learning model, a predictive data analysis computing entity can: (i) process an input behavioral timeseries data object for a monitored individual and/or an input biometric timeseries data object for a monitored individual in order to determine one or more activity patterns in the noted input data objects based at least in part on at least one of the input behavioral timeseries data object, the input biometric timeseries data object, and correlating the input biometric timeseries data object and the input behavioral timeseries data object, (ii) determine the improvement likelihood measures for the activity patterns in the noted input data objects to select a selected subset of the noted activity patterns (e.g., to select the top n activity patterns having the top n improvement likelihood measures, to select the activity patterns whose improvement likelihood measures satisfy an improvement likelihood measure, and/or the like), and (iii) present the selected subset of the noted activity patterns to an end user of the predictive data analysis computing entity.

In some embodiments, mappings between activity patterns and occurrence detection time window sets as described by the activity recommendation machine learning model can be used to infer activity patterns based at least in part on input behavioral timeseries data objects and input biometric timeseries data objects. In some embodiments, mappings between activity patterns and improvement likelihood measures can be used to select a selected subset of inferred detectivity patterns, where the inferred activity patterns may be inferred based at least in part on input behavioral timeseries data objects and input biometric timeseries data objects in accordance with mappings between activity patterns and occurrence detection time window sets. In some embodiments, a predictive data analysis computing entity is configured to provide access to the activity recommendation machine learning model, wherein the activity recommendation machine learning model is configured to determine, based at least in part on an input behavioral timeseries data object and an input biometric timeseries data object, a recommended activity pattern subset of the plurality of activity patterns.

At step/operation 1303, the predictive data analysis computing entity 106 provides user interface data for a recommended action user interface that describes the selected plurality of recommended actions to a client computing entity. In some embodiments, the client computing entity is configured to generate the recommended action user interface based at least in part on the user interface data for the recommended action user interface, and display the recommended action user interface to an end user of the client computing entity. In some embodiments, each recommended action describes performing activities corresponding to one or more detected activity patterns.

By using the above-described techniques, various embodiments of the present invention address technical challenges associated with correlating biometric data and behavioral data to perform predictive metabolic intervention by utilizing an activity recommendation machine learning model that maps each activity pattern to the occurrence detection time window set for the activity pattern and the improvement likelihood measure for the activity pattern, where activity patterns may be characterized by event patterns detected based on correlating biometric data and behavioral data, and the improvement likelihood measures may be determined based on biometric impact data. Using the noted techniques, various embodiments of the present invention generate activity recommendation machine learning models using computationally efficient operations configured to temporally align biometric timeseries data and behavioral timeseries data. In doing so, various embodiments of the present invention address technical challenges associated with efficiency and effectiveness of performing metabolic predictive data analysis

C. Predictive Metabolic Intervention using Prediction Window Encoding Machine Learning Models

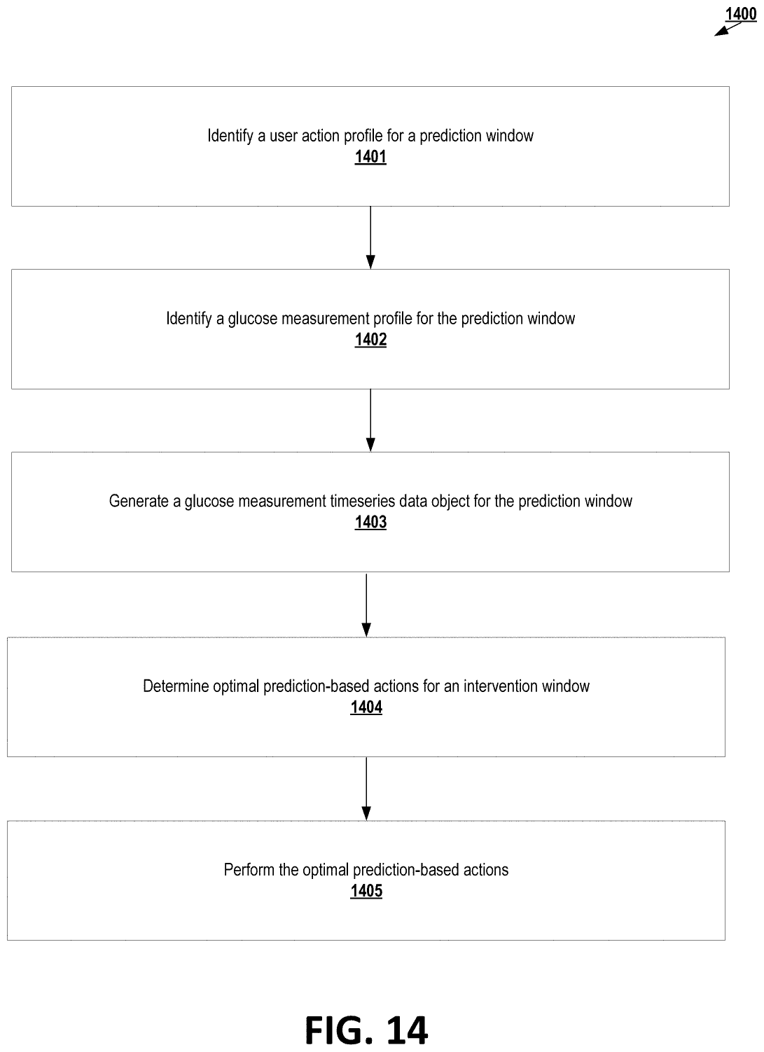

FIG. 14 is a flowchart diagram of an example process 1400 for performing predictive metabolic intervention using a prediction window encoding machine learning model, in accordance with some embodiments discussed herein. Via the various steps/operations of the process 1400, the predictive data analysis computing entity 106 can use joint encodings the glucose measurement time series data objects and the user activity profiles to determine recommended activities for monitored individuals associated with the glucose measurement time series data objects and the user activity profiles.

The process 1400 begins at step/operation 1401 when the predictive data analysis computing entity 106 receives a user activity profile for a prediction window. As described below, different prediction windows may have varied user activity profiles, which in turn complicates both integration of user activity data for those prediction windows into metabolic machine learning inferences as well as integration of glucose measurement data for those prediction windows into metabolic machine learning inferences. Aspects of prediction windows and user activity profiles are described in greater detail below.

A prediction window may describe a period of time whose respective user activity data and glucose measurement data may be used to determine appropriate prediction-based actions to perform during an intervention window subsequent to the prediction window. For example, in some embodiments, a prediction window may describe a particular period of time prior to a current time, where the user activity data and the physiological measurement data for the noted particular period of time may be used to determine appropriate prediction-based actions to perform during a subsequent period of time after the current time.

In some embodiments, the desired length of a period of time described by a prediction window is determined based at least in part on predefined configuration data, where the predefined configuration data may in turn be determined prior to runtime using user-provided data (e.g., system administration data), using rule-based models configured to determine optimal prediction window lengths based at least in part on patient activity data for the prediction window and/or based at least in part on glucose measurement data for the prediction window, using machine learning models configured to determine optimal prediction window lengths, and/or the like. In some embodiments, the desired length of a period of time described by a prediction window is determined based at least in part on configuration data that are dynamically generated at run-time using user-provided data (e.g., system administration data), using rule-based models configured to determine optimal prediction window lengths based at least in part on patient activity data for the prediction window and/or based at least in part on glucose measurement data for the prediction window, using machine learning models configured to determine optimal prediction window lengths, and/or the like. Examples of optimal lengths for periods of times described by prediction windows include twenty-four hours, ten days, two weeks, and/or the like.

As noted above, prediction windows may be associated with user activity data and glucose measurement data. The user activity data associated with a prediction window may describe one or more recorded user activity events associated with the prediction window. A recorded user activity event may describe attributes (e.g., occurrence, type, magnitude of glucose consumption, magnitude of predicted resulting glucose concentration increase, duration, frequency, and/or the like) of an activity performed by a monitored user, where a corresponding timestamp of the recorded user activity event may be within the period of time described by a corresponding prediction window. Examples of recorded user activity events for a prediction window may include bolus intake events associated with the prediction window, sleep events associated with the prediction window, exercise events associated with the prediction window, drug intake events associated with the prediction window, treatment usage events associated with the prediction window, and/or the like.

Given the preceding description of user event data associated with prediction windows, it should be apparent to a person of ordinary skill in the relevant technology that different prediction windows are not guaranteed to have the same number of recorded user activity events, let alone the same number of recorded user activity events of the same type or the same sequence of recorded user activity events of the same type. For example, a particular prediction window may be associated with four bolus intake events, while another prediction window may be associated with three bolus intake events. As another example, a particular prediction window may be associated with four bolus intake events each associated with a relatively high level of resulting glucose concentration increase (e.g., with four “heavy” meal intake sessions), while another prediction window may be associated with five bolus intake events associated with a relatively low level of resulting glucose concentration events (e.g., with five “light” meal intake sessions). As yet another example, a particular prediction window may be associated with three bolus intake events and two sleep events, while another prediction window may be associated with one bolus intake event and three sleep events.

A user activity profile for a corresponding prediction window may be configured to capture at least some aspects of the structural complexity of user activities of a particular prediction window. In some embodiments, a user activity profile describes recorded user activity events of a corresponding prediction window along with an activity order for the noted recorded user activity events. For example, a particular user activity profile may describe that a corresponding prediction window is associated with the following timeline of events: recorded user activity event A1 is performed prior to recorded user activity event A2, which is in turn performed prior to recorded user activity event A3. As another example, another user activity profile may describe that a corresponding prediction window is associated with the following timeline of events: (i) recorded user activity event A1 is performed closely before recorded user activity event A2, which is in turn performed closely before recorded user activity event A3; and (ii) recorded user activity event A4 is performed long after recorded user activity event A3. As yet another example, another user activity profile may describe that a corresponding prediction window is associated with the following timeline of events: (i) recorded user activity event A1 is performed two hours prior to recorded user activity event A2; (ii) recorded user activity event A2 is performed one hour prior to recorded user activity event A3; (iii) recorded user activity event A3 is performed thirty-four minutes prior to recorded user activity event A4; and (iv) recorded user activity event A4 is performed three hours prior to recorded user activity event A5. An example of a user activity profile is a bolus intake profile that describes sequential occurrence of one or more recorded user activity event. In some embodiments, the user activity profile includes a plurality of recorded user activity events associated with a prediction window that are separated by sufficient time from one another (e.g., separated by at least a length of time that is equal to the amount of time needed for glucose concentration levels of a monitored individual to return to a baseline glucose concentration level).

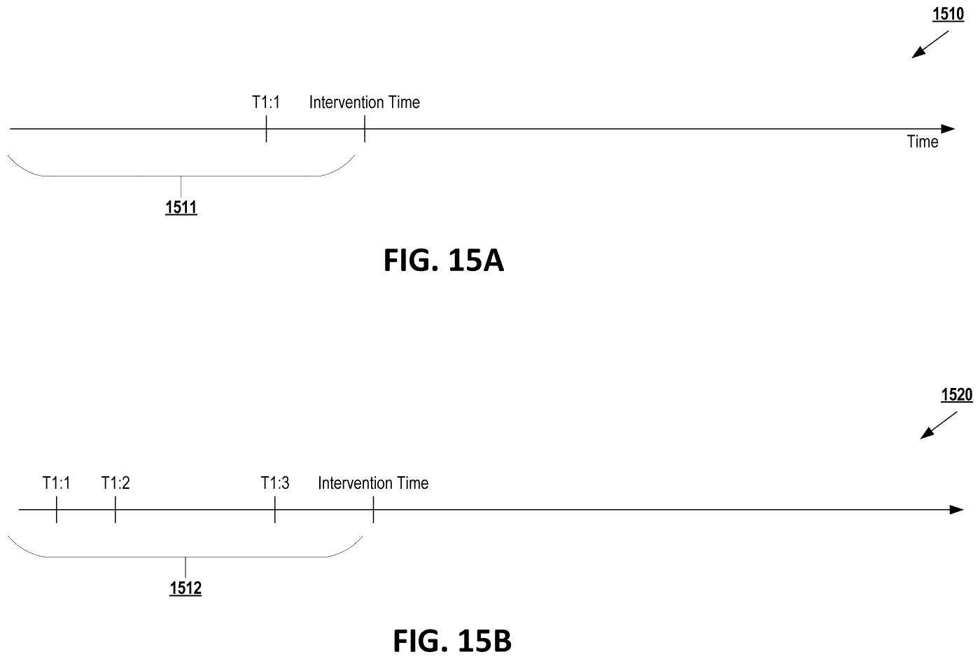

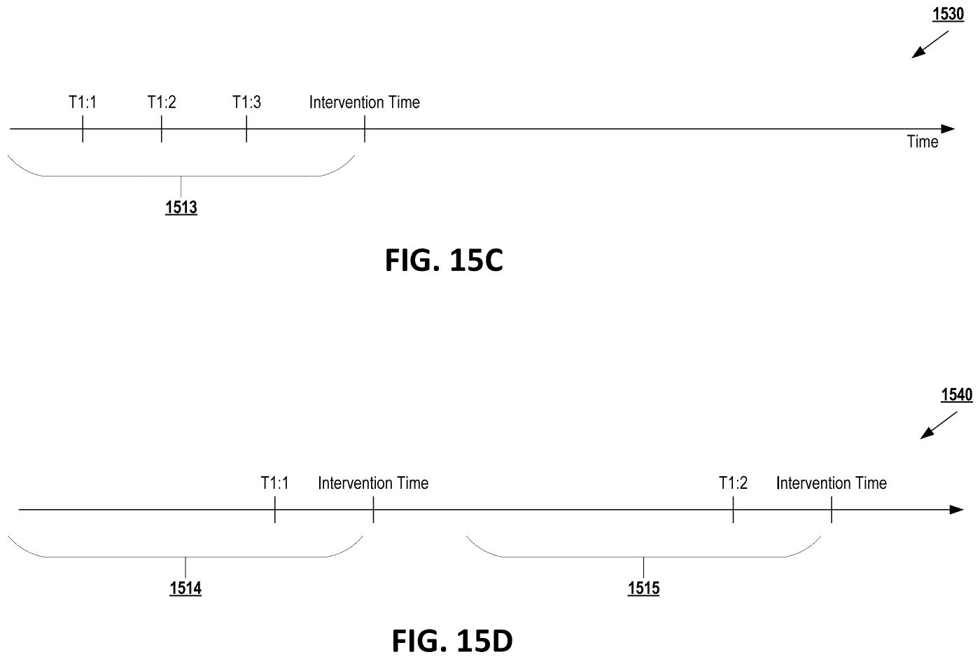

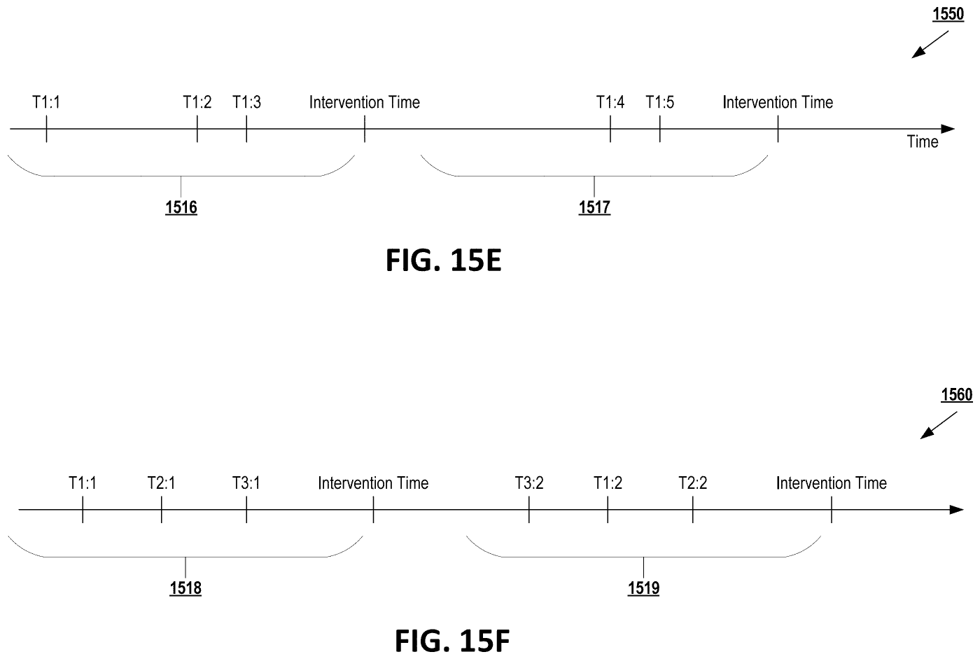

Operational examples of user activity profiles are depicted in FIGS. 15A-15F. As depicted in user activity profile 1510 of FIG. 15A, the prediction window 1511 includes the recorded user activity event A1:1, which describes a first occurrence of a first user activity type A1 (e.g., a heavy bolus intake, an insulin intake, and/or the like).

As further depicted in user activity profile 1520 of FIG. 15B, the prediction window 1512 includes recorded user activity event A1:1, which describes the first occurrence of the first user activity type A1, followed relatively closely by recorded user activity event A1:2, which describes a second occurrence of the first user activity type A1, followed relatively distantly by recorded user activity event A1:3, which describes a third occurrence of the first user activity type A1.

As further depicted in user activity profile 1530 of FIG. 15C, the prediction window 1513 includes recorded user activity event A1:1, which describes the first occurrence of the first user activity type A1, followed by recorded user activity event A2:1, which describes a first occurrence of the second user activity type A2, followed by recorded user activity event A3:1, which describes a first occurrence of the third user activity type A3.

As further depicted in user activity profile 1540 of FIG. 15D, the prediction window 1514 includes recorded user activity event A1:1, which describes the first occurrence of the first user activity type A1, while the prediction window 1515 includes recorded user activity event A2:1, which describes the second occurrence of the first user activity type A1.

As further depicted in user activity profile 1550 of FIG. 15E, prediction window 1516 includes recorded user activity event A1:1, which describes the first occurrence of the first user activity type A1, followed relatively distantly by recorded user activity event A1:2 which describes the second occurrence of the first user activity type A1, followed relatively closely by A1:3, which describes the third occurrence of the first user activity type A1; while prediction window 1517 includes recorded user activity event A1:4, which describes a fourth occurrence of the first user activity type A1, followed relatively closely by A1:5, which describes a fifth occurrence of the first user activity type A1.

As depicted in user activity profile 1560 of FIG. 15F, the prediction window 1518 includes recorded user activity event A1:1, which describes the first occurrence of the first user activity type A1, followed by recorded user activity event A2:1, which describes the first occurrence of the second user activity type A2, followed by A3:1, which describes a first occurrence of a third user activity type A3; while prediction window 1519 includes recorded user activity event A3:2, which describes a second occurrence of the third user activity type A3, followed by recorded user activity event A1:2, which describes the second occurrence of the first user activity type A1, followed by recorded user activity event A2:2, which describes a second occurrence of the second user activity type A2.

At step/operation 1402, the predictive data analysis computing entity 106 identifies a glucose measurement profile for the prediction window, where the glucose measurement profile describes one or more recorded glucose measurements associated with the prediction window (e.g., a portion of all of the recorded glucose measurements associated with the prediction window, all of the recorded glucose measurements associated with the prediction window, and/or the like). For example, the glucose measurement profile for a particular prediction window may describe one or more glucose measurements that were recorded during the particular time period associated with the prediction window by a glucose monitoring computing entity 101 and that were deemed statistically significant enough to transmit to the predictive data analysis computing entity 106.

In some embodiments, a glucose measurement profile for a corresponding prediction window is a data object that describes one or more glucose concentration measurements for the prediction window, where each corresponding timestamp for a glucose concentration measurement of the one or more glucose concentration measurements falls within a period of time described by the prediction window. In some embodiments, the timestamp of a glucose concentration measurement is determined based at least in part on a measurement time of the glucose concentration measurement. In some embodiments, a timestamp of a glucose concentration measurement is determined based at least in part on an adjusted measurement time of the glucose concentration measurement, wherein the adjusted measurement time may be determined by adjusting the measurement time of the glucose concentration measurement by a glucose concentration peak interval. In some embodiments, the glucose concentration measurements described by the glucose measurement profile may be determined using continuous glucose monitoring.

Aspects of various embodiments of the present invention determine recording time of glucose measurements during the time period associated with a prediction window to occurrence time of particular recorded user activity events during the noted time period. Thus, in some embodiments, each recorded glucose measurement of the one or more recorded glucose measurements is associated with a related subset of the one or more recorded user activity events. For example, in some embodiments, the predictive data analysis computing entity 106 may record a glucose measurement after each recorded user activity event (e.g., after each bolus intake event) during the prediction window. In some embodiments, the predictive data analysis computing entity 106 may record a glucose measurement after each n consecutive bolus intake events during the prediction window, where the value of n may be a predefined value or a generated value (e.g., a pre-runtime-generated value or a runtime-generated value), such as a value determined using a trained machine learning model. In some embodiments, the predictive data analysis computing entity 106 may record a glucose measurement after each bolus intake event whose predicted resulting glucose concentration increase exceeds a threshold predicted resulting glucose concentration increase (e.g., after a meal intake event deemed “heavy” enough). In some embodiments, by linking the time of glucose concentration measurement recordings to timing of user activity recordings, various embodiments of the present invention cause the diversity between user activity profiles of various prediction windows to in turn cause a diversity between physiological measurement profiles of various prediction windows, as physiological measurements are recorded based at least in part on occurrence of related events that are in turn defined to include one or more user activities.

At step/operation 1403, the predictive data analysis computing entity 106 generates a glucose measurement timeseries data object for the prediction window based at least in part on the user activity profile and the glucose measurement profile. In some embodiments, the predictive data analysis computing entity 106 combines the user activity profile and the glucose measurement profile in order to generate a representation of the recorded glucose measurements described by the glucose measurement profile that describes temporal relationships between the noted recorded glucose concentration measurements.

In some embodiments, a glucose measurement timeseries data object describes selected recorded glucose concentration measurements associated with a corresponding prediction window, where the selected recorded glucose concentration measurements are deemed related to (e.g., have timestamps that occur within a predefined time interval subsequent to, such as within 3-5 hours subsequent to) at least one recorded user activity event of a user activity profile. For example, a glucose concentration measurement timeseries data object may describe that a corresponding prediction window is associated with the following timeline of selected glucose concentration measurements: recorded glucose measurement M1 occurs prior to recorded glucose measurement M2, which is in turn performed prior to recorded glucose measurement M3. As another example, another glucose concentration measurement timeseries data object may describe that a corresponding prediction window is associated with the following timeline of selected glucose concentration measurements: (i) recorded glucose measurement M1 is performed closely before recorded glucose measurement M2, which is in turn performed closely before recorded glucose measurement M3; and (ii) recorded glucose measurement M4 is performed long after recorded glucose measurement M4. As yet another example, another glucose concentration measurement timeseries data object may describe that a corresponding prediction window is associated with the following timeline of selected glucose concentration measurements: (i) recorded glucose measurement M1 is performed three hours prior to recorded glucose measurement M2; (i) recorded glucose measurement M2 is performed two hours prior to recorded glucose measurement M3; (iii) recorded glucose measurement M3 is performed thirty-eight minutes prior to recorded glucose measurement M4; and (iv) recorded glucose measurement M4 is performed two hours prior to recorded glucose measurement M5. In some embodiments, the measurement timeseries data object describes the recorded glucose measurements along with one or more extrapolated glucose measurements inferred using one or more temporal extrapolation techniques to fill in the gaps between the noted recorded glucose concentration measurements. In some embodiments, a measurement order of selected glucose concentration measurements as described by a glucose measurement timeseries data object may be determined based at least in part on a temporal relationship of each timestamp associated with a selected glucose concentration measurement that is included in the glucose measurement timeseries data object.

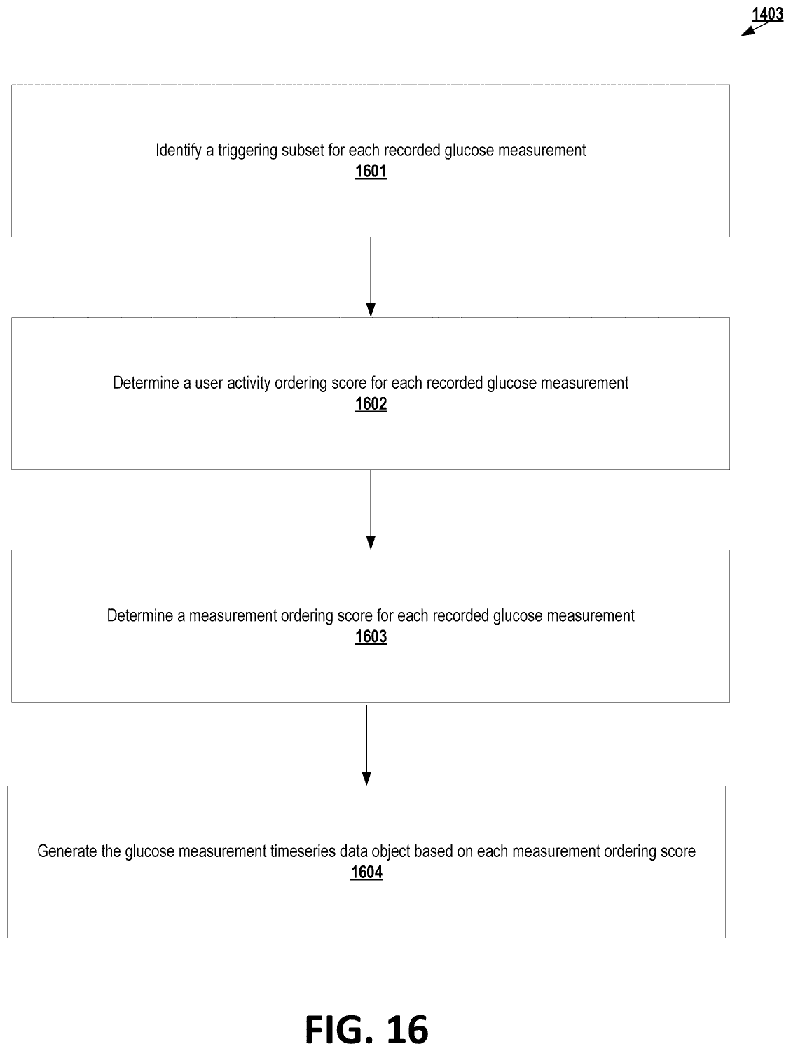

In some embodiments, step/operation 1403 may be performed in accordance with the process depicted in FIG. 16. As depicted in FIG. 16, the depicted process begins at step/operation 1601 when the predictive data analysis computing entity 106 identifies the related subset for each recorded glucose measurement.

The related subset for a recorded glucose measurement may describe a group of one or more recorded user activity events for a respective prediction window, where the recorded occurrence of the noted group of one or more recorded user activity events has caused a monitoring system to record a glucose concentration measurement in accordance with configuration data about appropriate timing of glucose concentration measurements. For example, the related subset of a corresponding recorded glucose measurement may correspond to one bolus intake event, one bolus intake event of a requisite nutritional energy, a required number of bolus intake events, one sleeping event, and/or the like.

At step/operation 1602, the predictive data analysis computing entity 106 determines a user activity ordering score for each recorded glucose measurement based at least in part on the precedence of at least one recorded user activity event in the related subset for the recorded glucose measurement according to the activity order of the user activity profile. For example, the predictive data analysis computing entity 106 may determine the user activity ordering score for a recorded glucose measurement based at least in part on the precedence of the latest-occurring recorded user activity event in the related subset for the recorded glucose measurement according to the activity order of the user activity profile. As another example, the predictive data analysis computing entity 106 may determine the user activity ordering score for a recorded glucose measurement based at least in part on the precedence of the most-related recorded user activity event in the related subset for the recorded glucose measurement according to the activity order of the user activity profile. As yet another example, if a recorded glucose measurement is associated with a sole recorded user activity event, the predictive data analysis computing entity 106 may determine the user activity ordering score for a recorded glucose measurement based at least in part on the precedence of the sole recorded user activity event in the related subset for the recorded glucose measurement according to the activity order of the user activity profile.

At step/operation 1603, the predictive data analysis computing entity 106 determines a measurement ordering score for each recorded glucose measurement based at least in part on the user activity recording score for the recorded glucose measurement. In some embodiments, the predictive data analysis computing entity 106 may assign the lowest possible measurement ordering score (i.e., the measurement ordering score that causes a corresponding recorded glucose measurement to be identified as the first-ordered recorded glucose measurement according to the measurement order) to the recorded glucose measurement having the lowest user activity ordering score among the user activity ordering scores of the recorded glucose measurements. In some embodiments, the predictive data analysis computing entity 106 may assign the second-lowest possible measurement ordering score (i.e., the measurement ordering score that causes a corresponding recorded glucose measurement to be identified as the second-ordered recorded glucose measurement according to the measurement order) to the recorded glucose measurement having the second-lowest user activity ordering score among the user activity ordering scores of the recorded glucose measurements. In some embodiments, the predictive data analysis computing entity 106 may assign the third-lowest possible measurement ordering score (i.e., the measurement ordering score that causes a corresponding recorded glucose measurement to be identified as the third-ordered recorded glucose measurement according to the measurement order) to the recorded glucose measurement having the third-lowest user activity ordering score among the user activity ordering scores of the recorded glucose measurements, and so on.

At step/operation 1604, the predictive data analysis computing entity 106 generates the glucose measurement timeseries data object for the prediction window based at least in part on each measurement ordering score for a recorded glucose measurement. In some embodiments, the predictive data analysis computing entity 106 adopts a particular ordering of the recorded glucose measurements in accordance with the measurement ordering scores and then generates the glucose measurement timeseries data object as a data object that is configured to describe the recorded glucose measurements in accordance with the noted measurement order.

Returning to FIG. 14, at step/operation 1404, the predictive data analysis computing entity 106 determines one or more recommended prediction-based actions for an intervention window subsequent to the prediction window based at least in part on the glucose measurement timeseries data object and the user activity profile. In some embodiments, the predictive data analysis computing entity 106 causes a machine learning framework to process the on the glucose measurement timeseries data object and the user activity profile to generate a classification score for each candidate prediction-based action of one or more candidate prediction-based actions and determine the one or more recommended prediction-based actions based at least in part on a subset of the candidate prediction-based actions whose classification score exceeds a threshold classification score. In some embodiments, the predictive data analysis computing entity 106 causes a machine learning framework to process the on the glucose measurement timeseries data object and the user activity profile to generate a classification score for each candidate prediction-based action of one or more candidate prediction-based actions and determine the recommended prediction-based actions based at least in part on top n candidate prediction-based actions having the highest classification score.

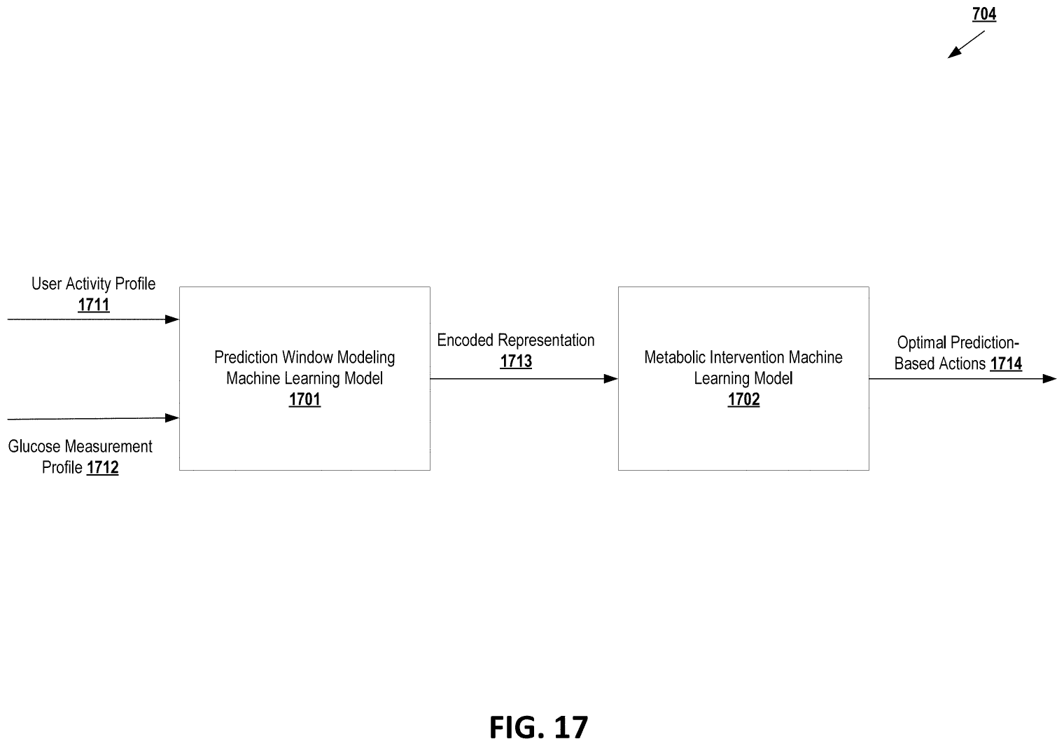

In some embodiments, step/operation 1404 may be performed in accordance with the process depicted in FIG. 17. The process depicted in FIG. 17 begins when a prediction window encoding machine learning model 1701 processes the glucose measurement timeseries data object 1711 and the user activity profile 1712 in order to generate an encoded representation 1713 for the prediction window. Aspects of prediction window encoding machine learning models and encoded representations for prediction windows are described in greater detail below.

A prediction window encoding machine learning model may be a machine learning model that is configured to generate a fixed-length representation of a prediction window that integrates the user activity data for the particular prediction window and the glucose measurement data for the particular prediction window. For example, the prediction window encoding machine learning model may be configured to generate a fixed-length representation of a prediction window that integrates the user activity profile for the prediction window and the glucose measurement profile for the prediction window. Examples of prediction window encoding machine learning models include encoder machine learning models, such as autoencoder machine learning models, variational autoencoder machine learning models, encoder machine learning models that include one or more recurrent neural networks such as one or more Long Short Term Memory units, and/or the like.

In some embodiments, the prediction window encoding machine learning model may generate a fixed-length representation of a particular prediction window that integrates, in addition to the user activity data for a particular prediction window and the glucose measurement data for a particular prediction window, at least one of the following: (i) a measure of one or more exogenous glucose infusion rates during the prediction window, (ii) a measure of one or more insulin-dependent glucose uptake coefficients during the particular prediction window, (iii) a measure of one or more hepatic glucose production rates during the particular prediction window, (iv) a measure of insulin degradation rates during the particular prediction window, (v) a measure of one or more maximal insulin secretion rates during the particular prediction window, (vi) a measure of one or more insulin-independent glucose uptake rates during the particular prediction window, (vii) a measure of one or more insulin secretion accelerations during the particular prediction window, (viii) a measure of one or more insulin secretion time delays during the particular prediction window, and (ix) a measure of one or more glucose concentration peak intervals during the particular prediction window.

In some embodiments, an encoded representation for a prediction window is the fixed-length representation for the particular prediction window that is generated by processing the user activity data for the particular prediction window and the glucose measurement data for the particular prediction window. In some embodiments, in addition to the user activity data for a particular prediction window and the glucose measurement data for a particular prediction window, the fixed-length representation of a particular prediction window may integrate at least one of the following: (i) a measure of one or more exogenous glucose infusion rates during the prediction window, (ii) a measure of one or more insulin-dependent glucose uptake coefficients during the particular prediction window, (iii) a measure of one or more hepatic glucose production rates during the particular prediction window, (iv) a measure of insulin degradation rates during the particular prediction window, (v) a measure of one or more maximal insulin secretion rates during the particular prediction window, (vi) a measure of one or more insulin-independent glucose uptake rates during the particular prediction window, (vii) a measure of one or more insulin secretion accelerations during the particular prediction window, (viii) a measure of one or more insulin secretion time delays during the particular prediction window, and (ix) a measure of one or more glucose concentration peak intervals during the particular prediction window.

As further depicted in FIG. 17, a metabolic intervention machine learning model 1702 processes the encoded representation 1713 for the prediction window in order to determine one or more recommended prediction-based actions 1714 for an intervention window subsequent to the prediction window. Aspects of metabolic intervention machine learning models are described in greater detail below. In some embodiments, the metabolic intervention machine learning model 1702 (alone or in combination with the prediction window encoding machine learning model 1701) may be trained in accordance with the techniques for training machine learning models that are discussed in Exhibit A.

A metabolic intervention machine learning model may be a machine learning model that is configured to process the encoded representation for a prediction window in order to determine one or more recommended prediction-based actions for an intervention window subsequent to the prediction window. In some embodiments, the metabolic intervention machine learning model is a supervised machine learning model (e.g., a neural network model) trained using labeled data associated with one or more ground-truth prediction windows (e.g., one or more previously-treated prediction windows), where the supervised machine learning model is configured to generate a classification score for each candidate prediction-based action of one or more candidate prediction-based actions and use each classification score for a candidate prediction-based action to determine the recommended prediction-based actions. In some embodiments, the metabolic intervention machine learning model is an unsupervised machine learning model (e.g., a clustering model), where the unsupervised machine learning model is configured to map encoded representation of the prediction window into a multi-dimensional space including mappings of encoded representations of one or more ground-truth prediction windows in order to determine a selected subset of the ground-truth prediction windows whose encoded representation mapping is deemed sufficiently close to the encoded representation mapping of the particular prediction window, and use information about treatment of the selected subset of the ground-truth prediction windows to determine the recommended prediction-based actions.

In some embodiments, the metabolic intervention machine learning model may be configured to process the encoded representation for a prediction window to determine a metabolic value for each candidate prediction-based action given the prediction window. A metabolic value may be any indicator of metabolic health derived, at least in part, from glucose measurements. Nonlimiting examples of metabolic values may include physiological measures such as insulin sensitivity and/or beta cell capacity. Further nonlimiting examples of metabolic values may include area under a curve of glucose readings generated over time, the slope of such readings, or the variability of such readings. In some embodiments, metabolic values may comprise an amount or time necessary for a particular response. For example, a metabolic value may comprise the maximum amount of glucose that an individual can dispose of (e.g., return to a baseline glucose concentration) within a given amount of time. As another example, a metabolic value may comprise an amount of time necessary to dispose of a given quantity of glucose.

Examples of candidate prediction-based actions include treatment recommendation actions (e.g., generating a computer-presented notification, generating user interface data for a user interface, and/or the like). As used herein, a treatment, referred to in the singular, may include one or more treatments. For example, a treatment may include one drug or multiple drugs. Nonlimiting examples of drugs include biguanides, GLP-1, SGLT-2, DPP-4, sulfonylurea, basal insulin, or bolus insulin. The distinguishing feature of a treatment may be a characteristic of drug administration. For example, a first treatment and a second treatment may both comprise the same drug but differ in dosage or in the schedule on which the drug is administered. A treatment need not be a drug or drug combination. A treatment may comprise one or more non-drug elements such as a behavioral regimen. For example, behavioral regimens may comprise changes in diet including limitation of overall calories, limitation of particular nutrients such as carbohydrates, or fasting for particular durations. Fasting regimens may comprise a single time period of fasting, or an ongoing schedule of fasting periods interspersed with non-fasting periods. Nonlimiting examples of fasting schedules may include (fasting/feeding time): 12 hours/12 hours; 10 hours/14 hours; 8 hours/16 hours; and/or 5 days/2 days. As another example, a treatment may comprise cessation or withdrawal of drug or other treatment received by an individual.

Returning to FIG. 14, at step/operation 1405, the predictive data analysis computing entity 106 causes the one or more prediction-based actions to be performed. For example, the predictive data analysis computing entity 106 may be configured to generate one or more physician alerts and/or one or more healthcare provider alerts based at least in part on the glucose-insulin predictions. As another example, the predictive data analysis computing entity 106 may be configured to generate one or more automated physician appointments, automated medical notes, automated prescription recommendations, and/or the like based at least in part on glucose-insulin predictions determined based at least in part on the encoded representations of prediction windows. As yet another example, the predictive data analysis computing entity 106 may be configured to enable an end-user device to display a user interface, where the user interface has been generated based at least in part on the glucose-insulin predictions determined based at least in part on the encoded representations of prediction windows.

In some embodiments, performing the one or more prediction-based actions includes combining data for a patient that has type II diabetes from a continuous glucose monitoring device with data derived from conventional wearable devices like a Fitbit or cell phone sensors. The combined data is then used to identify patterns in the blood glucose readings as they relate to the other data. In some embodiments, the predictive data analysis computing entity 106 also has access to and uses the patient's phenotype data and other patient information (e.g., demographic info) for the identification of the patterns.

For example, the predictive data analysis computing entity 106 may determine that glucose spikes occur at regular times of day. As another example, the predictive data analysis computing entity 106 may identify a pattern between the timing of eating and glucose levels. As fasting drives remission, the predictive data analysis computing entity 106 may attempt to manage the patient's fasting to prevent hypoglycemia. The predictive data analysis computing entity 106 can further choose between different fasting regimens based at least in part on the real-time feedback from the continuous glucose monitoring device. The predictive data analysis computing entity 106 can also identify patterns in the way that the timing of drugs (e.g., morning vs. evening) affects the patient's glucose levels based at least in part on the real-time feedback from the continuous glucose monitoring device.

In some embodiments, based at least in part on the identified glucose patterns, the predictive data analysis computing entity 106 may suggest one or more micro-interventions to the user, such as by taking a walk or eating a particular food. The presentation of the noted suggestions may occur through a coaching portal. Because many micro-interventions may be relevant or triggered by the data being reported for the patient at any given time, the predictive data analysis computing entity 106 may prioritize the triggered micro-interventions so that the patient is not inundated with too many micro-interventions at once. The predictive data analysis computing entity 106 may also suggest a treatment to the person's doctor through a specialist portal. The doctor may then verify the treatment and perform the treatment for the patient.

Furthermore, the predictive data analysis computing entity 106 may also provide a benefits portal where the patient may earn points toward rewards by participating in the monitoring program and performing the suggested micro-interventions. In some embodiments, the predictive data analysis computing entity 106 may provide access to the program through a benefits portal. The reward may be a monetary reward, such as by waiving a co-pay for a next doctor visit. The micro-interventions may be related to performing physical activity, such as walking. The predictive data analysis computing entity 106 can verify that the suggested physical activity occurred using the wearable sensors. The patient may also report the performance of other micro-interventions manually where there is no corresponding sensor data, such as when the patient eats a particular food that was suggested by the predictive data analysis computing entity 106 in a micro-intervention. In some embodiments, the benefits program may include a standard tier and a premium tier. The premium tier may have greater rewards than the standard tier. The predictive data analysis computing entity 106 may further select a tier for a patient based at least in part on the points accrued by the patient.

In some embodiments, performing the one or more prediction-based actions comprises generating an insulin sensitivity prediction based at least in part on at least one of an maximal insulin secretion rate value and an insulin secretion acceleration value; and determining, based at least in part on the insulin sensitivity measure, an exogenous insulin need determination. In some of the noted embodiments, performing the one or more prediction-based actions further comprises, in response to determining a positive exogenous insulin need determination, generating one or more automated medical alarms. In some of the noted embodiments, performing the one or more prediction-based actions further comprises, in response to determining a positive exogenous insulin need determination, causing the automated insulin delivery computing entity 102 to perform an automated exogenous insulin injection into the bloodstream of the corresponding monitored individual. In some of the noted embodiments, performing the one or more prediction-based actions further comprises, in response to determining a positive exogenous insulin need determination, causing an automated medical response such as arrangement of ambulance services for the corresponding monitored individual.

In some embodiments, performing the one or more prediction-based actions comprises generating a glucose-insulin prediction for a monitored individual and performing an action based at least in part on the glucose-insulin prediction. A glucose-insulin prediction may describe a conclusion about one or more functional properties of the glucose-insulin endocrine metabolic regulatory system of a corresponding monitored individual. For example, the predictive data analysis computing entity 106 may determine an insulin sensitivity prediction based at least in part on at least one of the maximal insulin secretion rate value and the insulin secretion acceleration value. In some embodiments, if the maximal insulin secretion rate parameter is higher than an expected amount, a computer system may determine that the insulin-dependent glucose-utilizing cells of the monitored individual have developed abnormal levels of insulin sensitivity, which in turn may be used to facilitate an automated diagnosis of type-2 diabetes. As another example, the predictive data analysis computing entity 106 may detect a potential liver problem based at least in part on an abnormally hepatic glucose production parameter. As yet another example, the predictive data analysis computing entity 106 may detect a potential nervous system problem if the insulin-independent glucose uptake rate parameter is abnormally low.

In some embodiments, the predictive data analysis computing entity 106 is configured to identify a user activity profile for a prediction window, wherein the user activity profile describes one or more recorded user activity events as well as an activity order for the recorded user activity events; identify a glucose measurement profile for the prediction window, wherein the glucose measurement profile describes one or more recorded glucose measurements associated with the prediction window; generate a glucose measurement time series data object for the prediction window based at least in part on the user activity profile and the glucose measurement profile, wherein the glucose measurement time series data object describes a subset of the one or more glucose measurements that are deemed related to the one or more recorded user activity events and indicates a measurement order for the one or more glucose measurements; process the glucose measurement time series data object and the user activity profile using a prediction window encoding machine learning model in order to generate an encoded representation for the prediction window; and process the encoded representation using a metabolic intervention machine learning model in order to determine one or more recommended prediction-based actions for an intervention window subsequent to the prediction window and cause performance of the one or more recommended prediction-based actions.

Accordingly, various embodiments of the present make substantial contributions to the field of treating metabolic dysfunctions. Some of the methods described herein use one or more processors to select a treatment to improve the metabolic health of an individual using glucose readings from an individual obtained after the individual has consumed one or more boluses of known content. The one or more processors may use the glucose readings and a machine learning model to predict a metabolic value. The one or more processors may select the treatment from among a plurality of treatments where the selected treatment is associated with the predicted metabolic value that is closest to an optimal value. By utilizing the noted techniques, various embodiments of the present invention improve treatment of individuals suffering from metabolic dysfunctions.

Moreover, using the above-described techniques, various embodiments of the present invention address technical challenges related to efficiency and effectiveness of performing metabolic predictive data analysis. Some of the efficiency and effectiveness challenges associated with performing metabolic predictive data analysis results from the fact that user activity data (e.g., bolus intake data) and glucose measurement data associated with different predictive windows may be variable in size. This causes challenges for existing machine learning models that expect predictive inputs of a predefined format and structure. Moreover, machine learning models that accept variable-size inputs, such as sequential processing models including recurrent neural networks, are excessively computationally resource-intensive.

### VI. CONCLUSION

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented for predictive metabolic intervention, the computer-implemented method comprising:
identifying, by a processor, a behavioral timeseries data object associated with a plurality of behavioral time windows;
identifying, by the processor, a biometric timeseries data object associated with a plurality of biometric time windows;
for each biometric time window, determining, by the processor, a desired outcome indicator based at least in part on the biometric timeseries data object;
determining, by the processor, a plurality of activity patterns based at least in part on at least one of the behavioral timeseries data object or the biometric timeseries data object, wherein:
each activity pattern is identified based at least in part on an occurrence detection time window set comprising at least one of a behavioral occurrence detection time window subset of the plurality of behavioral time windows or a biometric occurrence detection time window subset of the plurality of biometric time windows, and
each activity pattern is associated with a biometric impact subset of the plurality of biometric time windows;
for each activity pattern, determining, by the processor, an improvement likelihood measure based at least in part on each desired outcome indicator for a biometric time window that is in the biometric impact subset for the activity pattern;
generating, by the processor, an activity recommendation machine learning model, wherein the activity recommendation machine learning model maps each activity pattern to the occurrence detection time window set for the activity pattern and the improvement likelihood measure for the activity pattern; and
providing access, by the processor, to the activity recommendation machine learning model, wherein the activity recommendation machine learning model is configured to determine, based at least in part on an input behavioral timeseries data object and an input biometric timeseries data object, a recommended activity pattern subset of the plurality of activity patterns.

2. The computer-implemented method of claim 1, wherein:
the plurality of activity patterns comprises one or more biometric activity patterns, and
the occurrence detection time window set for each biometric activity pattern comprises the biometric occurrence detection time window subset for the biometric activity pattern.

3. The computer-implemented method of claim 1, wherein:
the plurality of activity patterns comprises one or more behavioral activity patterns, and
the occurrence detection time window set for each behavioral activity pattern comprises the behavioral occurrence detection time window subset for the behavioral activity pattern.

4. The computer-implemented method of claim 1, wherein:
the plurality of activity patterns comprises one or more behavioral-biometric activity patterns,
the occurrence detection time window set for each behavioral-biometric activity pattern comprises both the behavioral occurrence detection time window subset for the behavioral-biometric activity pattern and the biometric occurrence detection time window subset for the behavioral-biometric activity pattern, and
each behavioral-biometric activity pattern is determined based at least in part on one or more detected cross-timeseries correlations across the plurality of behavioral time windows and the plurality of biometric time windows.

5. The computer-implemented method of claim 1, wherein the behavioral timeseries data object is generated based at least in part on one or more recorded longitudinal observations of a corresponding individual across the plurality of behavioral time windows.

6. The computer-implemented method of claim 1, wherein:
the behavioral timeseries data object is generated based at least in part on each plurality of recorded observations for an individual of a plurality of individuals, and
each plurality of recorded observations for an individual is determined based at least in part on a plurality of observation time windows for the individual, and
the plurality of behavioral time windows comprises each plurality of observation time windows for an individual.

7. The computer-implemented method of claim 1, wherein the biometric timeseries data object is generated based at least in part on one or more recorded longitudinal observations of a corresponding individual across the plurality of biometric time windows.

8. The computer-implemented method of claim 1, wherein:
the biometric timeseries data object is generated based at least in part on each plurality of recorded observations for an individual of a plurality of individuals, and
each plurality of recorded observations for an individual is determined based at least in part on a plurality of observation time windows for the individual, and
the plurality of biometric time windows comprise each plurality of observation time windows for an individual.

9. The computer-implemented method of claim 1, wherein each desired outcome indicator for a biometric time window is a target time in range measure for the corresponding biometric time window.

10. An apparatus comprising at least one processor and at least one memory including computer program code is provided. In one embodiment, the at least one memory and the computer program code may be configured to, with the processor, cause the apparatus to:
identify a behavioral timeseries data object associated with a plurality of behavioral time windows;
identify a biometric timeseries data object associated with a plurality of biometric time windows;
for each biometric time window, determine a desired outcome indicator based at least in part on the biometric timeseries data object;
determine a plurality of activity patterns based at least in part on at least one of the behavioral timeseries data object or the biometric timeseries data object, wherein:
each activity pattern is identified based at least in part on an occurrence detection time window set comprising at least one of a behavioral occurrence detection time window subset of the plurality of behavioral time windows or a biometric occurrence detection time window subset of the plurality of biometric time windows, and
each activity pattern is associated with a biometric impact subset of the plurality of biometric time windows;
for each activity pattern, determine an improvement likelihood measure based at least in part on each desired outcome indicator for a biometric time window that is in the biometric impact subset for the activity pattern;
generate an activity recommendation machine learning model, wherein the activity recommendation machine learning model maps each activity pattern to the occurrence detection time window set for the activity pattern and the improvement likelihood measure for the activity pattern; and
provide access to the activity recommendation machine learning model, wherein the activity recommendation machine learning model is configured to determine, based at least in part on an input behavioral timeseries data object and an input biometric timeseries data object, a recommended activity pattern subset of the plurality of activity patterns.

11. The apparatus of claim 10, wherein:
the plurality of activity patterns comprise one or more biometric activity patterns, and
the occurrence detection time window set for each biometric activity pattern comprises the biometric occurrence detection time window subset for the biometric activity pattern.

12. The apparatus of claim 10, wherein:
the plurality of activity patterns comprise one or more behavioral activity patterns, and
the occurrence detection time window set for each behavioral activity pattern comprises the behavioral occurrence detection time window subset for the behavioral activity pattern.

13. The apparatus of claim 10, wherein:
the plurality of activity patterns comprise one or more behavioral-biometric activity patterns,
the occurrence detection time window set for each behavioral-biometric activity pattern comprises both the behavioral occurrence detection time window subset for the behavioral-biometric activity pattern and the biometric occurrence detection time window subset for the behavioral-biometric activity pattern, and
each behavioral-biometric activity pattern is determined based at least in part on one or more detected cross-timeseries correlations across the plurality of behavioral time windows and the plurality of biometric time windows.

14. The apparatus of claim 10, wherein the behavioral timeseries data object is generated based at least in part on one or more recorded longitudinal observations of a corresponding individual across the plurality of behavioral time windows.

15. The apparatus of claim 10, wherein:
the behavioral timeseries data object is generated based at least in part on each plurality of recorded observations for an individual of a plurality of individuals, and
each plurality of recorded observations for an individual is determined based at least in part on a plurality of observation time windows for the individual, and
the plurality of behavioral time windows comprise each plurality of observation time windows for an individual.

16. The apparatus of claim 10, wherein the biometric timeseries data object is generated based at least in part on one or more recorded longitudinal observations of a corresponding individual across the plurality of biometric time windows.

17. The apparatus of claim 10, wherein:
the biometric timeseries data object is generated based at least in part on each plurality of recorded observations for an individual of a plurality of individuals, and
each plurality of recorded observations for an individual is determined based at least in part on a plurality of observation time windows for the individual, and
the plurality of biometric time windows comprise each plurality of observation time windows for an individual.

18. The apparatus of claim 10, wherein each desired outcome indicator for a biometric time window is a target time in range measure for the corresponding biometric time window.

19. A computer program product may comprise at least one computer-readable storage medium having computer-readable program code portions stored therein, the computer-readable program code portions comprising executable portions configured to:
identify a behavioral timeseries data object associated with a plurality of behavioral time windows;
identify a biometric timeseries data object associated with a plurality of biometric time windows;
for each biometric time window, determine a desired outcome indicator based at least in part on the biometric timeseries data object;
determine a plurality of activity patterns based at least in part on at least one of the behavioral timeseries data object or the biometric timeseries data object, wherein:
each activity pattern is identified based at least in part on an occurrence detection time window set comprising at least one of a behavioral occurrence detection time window subset of the plurality of behavioral time windows or a biometric occurrence detection time window subset of the plurality of biometric time windows, and
each activity pattern is associated with a biometric impact subset of the plurality of biometric time windows;
for each activity pattern, determine an improvement likelihood measure based at least in part on each desired outcome indicator for a biometric time window that is in the biometric impact subset for the activity pattern;
generate an activity recommendation machine learning model, wherein the activity recommendation machine learning model maps each activity pattern to the occurrence detection time window set for the activity pattern and the improvement likelihood measure for the activity pattern; and
provide access to the activity recommendation machine learning model, wherein the activity recommendation machine learning model is configured to determine, based at least in part on an input behavioral timeseries data object and an input biometric timeseries data object, a recommended activity pattern subset of the plurality of activity patterns.

20. The computer program product of claim 19, wherein:
the plurality of activity patterns comprise one or more biometric activity patterns, and
the occurrence detection time window set for each biometric activity pattern comprises the biometric occurrence detection time window subset for the biometric activity pattern.
